(19) United States
(12) Patent Application Publication
(10) Pub. No.: US 2009/0246152 A1
(43) Pub. Date: Oct. 1, 2009

(54) NARACTIN COMPOSITIONS FOR THE INHIBITION OF REACTIVE OXYGEN SPECIES

(75) Inventors: Dale Kern, Hyde Park, UT (US); Christiaan Meadows, Lafayette, IN (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: Nu Skin International, Inc., Provo, UT (US)

(21) Appl. No.: 12/058,201

(22) Filed: Mar. 28, 2008

(51) Int. Cl. A61K 8/30; A61K 31/7034; A61K 31/60; A61K 36/31; A61K 36/07; A61K 36/53; A61K 36/28; A61K 36/752; A61P 17/00; A61Q 19/00; A61K 8/97; A61K 36/35; A61K 36/704; A61K 36/23; A61K 36/896; A61K 36/63; A61K 36/899; A61K 31/122

(52) U.S. Cl. 424/59; 514/35; 514/159; 424/755; 424/195.15; 424/765; 424/725; 424/736; 424/753; 424/750; 514/691; 514/763; 510/119

(57) ABSTRACT

The invention relates to agents for sequestering serum aging factors, and methods for using the same. More particularly, the invention relates to agents termed herein “Naractin” to denote any one of several naturally-occurring arNOX inhibitors either present in N. tazetta powder or capable of augmenting N. tazetta powder to an inhibitory level comparable to that of the fresh N. tazetta extracts, and to methods for using “Naractins” to prevent or treat disorders and complications of disorders resulting from cell damage caused by an aging-related isoform of NADH oxidase (arNOX). In one exemplary embodiment the agents of the invention comprise at least one naturally occurring Naractin. Such naturally occurring naractins are also capable of augmenting the anti-arNOX effect of other naturally occurring arNOX inhibitory agents.

### FIELD OF THE INVENTION

The invention relates to extracts of natural products useful in sequestering serum aging factors that may be administered internally or topically. More particularly, the invention relates to agents and compositions thereof for use cosmetically to inhibit or ameliorate aging-related oxidation and methods for their use as skin care products.

### BACKGROUND OF THE INVENTION

The plasma membrane NADH oxidase (NOX) is a unique cell surface protein with hydroquinone (NADH) oxidase and protein disulfide-thiol interchange activities that normally responds to hormone and growth factors. NOX (or CLOX) are a family of growth related proteins that are associated with aging cells. A hormone-insensitive and drug-responsive form of the NOX designated tNOX has been described that is specific for cancer cells. For example, see U.S. Pat. No. 5,605,810, which is incorporated herein by reference in its entirety.

The aging-related isoform of NADH oxidase (arNOX) is a member of this family of proteins. The circulating form of arNOX increases markedly in human sera and in lymphocytes of individuals, especially between the ages of 30 to 65. The arNOX protein is uniquely characterized by an ability to generate superoxide radicals, which may contribute significantly to aging-related changes including atherogenesis and other action-at-a-distance aging phenomena. Activity of arNOX in aging cells and in sera has been described previously. See, for example, PCT Pub. App. No. WO 00/57871, which is incorporated by reference in its entirety herein.

This model of the effects of arNOX is consistent with the Mitrochondrial Theory of Aging, which holds that during aging, increased reactive oxygen species in mitochondria cause mutations in the mitochondrial DNA and damage mitochondrial components, resulting in senescence. The mitochondrial theory of aging proposes that accumulation of spontaneous somatic mutations of mitochondrial DNA (mtDNA) leads to errors of mtDNA encoded polypeptide chains. (Manczak M et al., J Neurochem. February 2005; 92(3):494-504). These errors, occurring in mtDNA encoded polypeptide chains, are stochastic and randomly transmitted during mitochondrial and cell division. The consequence of these alterations is defective oxidative phosphorylation. Respiratory chain defects may become associated with increased oxidative stress amplifying the original damage (Ozawa, 1995, Biochim. Biophys. Acta 1271:177-189; and Lenaz, 1998, Biochim. Biophys. Acta 1366:53-67). In this view, therefore, mutated mitochondrial DNA, despite being present only in very small quantities in the body, may be the major generator of oxidative stress.

Where accumulation of somatic mutations of mtDNA leads to defective oxidative phosphorylation a plasma membrane oxido-reductase (PMOR) system has been suggested to augment survival of mitochondrially deficient cells through regeneration of oxidized pyridine nucleotide. (de Grey, 1997, BioEssays 19:16 1-166; de Grey, 1998, Anti-Aging Med. 1:53-66; Yoneda et al, 1995, Biochem. Biophys. Res. Comm, 209:723-729; Schon et al., 1996, Cellular Aging and Cell Death, Wiley and Sons, New York, pp. 19-34; Ozawa, 1997, Physiol. Rev. 77:425-464; and Lenaz, 1998, BioFactors 8:195-204). A model to link accumulation of lesions in mtDNA to extracellular responses, such as the oxidation of lipids in low density lipoprotein (LDLs) and the attendant arterial changes, was first proposed with rho° cells (Larm et al., 1994, Biol. Chem. 269:30097-30100; Lawen et al., 1994, Mol. Aspects. Med. 15:s13-s27; de Grey, 1997, BioEssays 19:161-166; and de Grey, 1998, Anti-Aging Med. 1:53-66). Similar studies have been conducted with transformed human cells in culture. (Vaillant et al., 1996, Bioenerg. Biomemb. 28:53 1-540).

Under conditions where plasma membrane oxidoreductase (PMOR) is overexpressed electrons are transferred from NADH to external acceptors by a defined electron transport chain, resulting in the generation of reactive oxygen species (ROS) at the cell surface. Such cell surface-generated ROS may then propagate an aging cascade originating in mitochondria to both adjacent cells as well as to circulating blood components such as low density lipoproteins. See PCT Pub. App. No. WO 00/57871 incorporated by reference herein in its entirety.

Consequently, there is a need to find agents that reduce the ability of arNOX to generate reactive oxygen species (ROS) for the purposes of reducing or treating the resultant physiological conditions, such as oxidation of lipids in low density lipoprotein (LDLs) and attendant arterial changes. The arNOX activity of aging cells has been shown to be inhibited by naturally occurring agents such as, co-enzyme Q (ubiquinone). See PCT Pub. App. No WO 00/57871, WO 01/72318, and WO 01/72319, the disclosures of which are incorporated herein by reference in their entirety. However, the use of co-enzyme Q is not completely satisfactory for several reasons: it is costly, it oxidizes easily losing its efficacy, and preparations containing coenzyme Q must be specially packaged to prevent loss of function. Thus, while some agents and methods currently exist, which may inhibit arNOX activity, challenges still exist. Accordingly, it would be an improvement in the art to augment or even replace previously disclosed agents and techniques with the agents and techniques that inhibit arNOX but that are also non-toxic and naturally occurring.

The skin in particular is vulnerable to damage by reactive oxygen species. The skin is composed of two major layers. The stratum corneum, or epidermis, is the top layer and forms a protective covering for the skin and controls the flow of water and substances in and out of the skin. The dermis is the lower level of the skin and provides the strength, elasticity and the thickness to the skin. The main cell type of the dermis is fibroblasts, which are responsible for synthesis and secretion of all the dermal matrix components such as collagen, elastin and glycosaminoglycans. Collagen provides the strength, elastin the elasticity, and glycosaminoglycans the moistness and plumpness of the skin.

In addition to being damaged by reactive oxygen species the skin is subject to various damaging stressors. The skin may be damaged or abused by many factors in the environment. Some are naturally occurring such as UV radiation from the sun, wind and even mechanical insults such as cuts, scrapes and the like. Other, man-made insults also occur daily. These include the use of soaps, emulsifier-based cosmetics, hot water, organic solvents, air conditioning and central heating. Further, other insults to the skin may result from or be part of dermatological disorders or the normal aging process (chronoaging), which may be accelerated by exposure of skin various external stressors (e.g. photoaging).

Everyone's skin ages with time. In modern society, however, people live longer and the normal effects of aging have an opportunity to accumulate. Such effects may be purely cosmetic, such as the increase in wrinkles or “age spots” or they may have an impact on health such as the incidence of skin cancer due to exposure to UV light. As people age, the skin becomes thinner, the connective tissue of the skin, collagen and elastin changes causing the skin to loose firmness and become dry. Also, the sweat and oil glands of the skin become less active thereby causing the skin to lose moisture and dry out. Further, blood vessels in the skin become more fragile so that they rupture and leak into the skin.

Symptoms of aging skin include dryness, itchiness, thinning or thickening of the skin, wrinkles and fine lines, areas of hyperpigmentation commonly referred to as liver spots and areas underneath the skin where blood vessels have ruptured (telangietasias).

“Anti-aging” cosmetic and medical products, which treat or delay the visible signs of actual aging and weathered skin such as wrinkles, lines, sagging, hyperpigmentation and age spots are desirable. However, most cosmetic or medicinal products do not address the bases of such symptoms, e.g., the production and buildup of arNOX related radicals derived from ROS. Accordingly, there is a demand for effective natural skin treatments and preventative compositions and methods for using the same.

### SUMMARY OF THE INVENTION

The invention relates to agents for sequestering serum aging factors, and methods for using the same. More particularly, the invention relates to agents termed herein “Naractin” to denote any one of several naturally-occurring arNOX inhibitors either present in N. tazetta powder or capable of augmenting N. tazetta powder to an inhibitory level comparable to that of the fresh N. tazetta extracts, and to methods for using “Naractins” to prevent or treat disorders and complications of disorders resulting from cell damage caused by an aging-related isoform of NADH oxidase (arNOX). In one exemplary embodiment the agents of the invention comprise at least one naturally occurring Naractin. Such naturally occurring naractins are also capable of augmenting the anti-arNOX effect of other naturally occurring arNOX inhibitory agents.

Therefore, in one exemplary embodiment the invention includes a topical composition useful for ameliorating the effects of aging comprising an effective amount of at least one arNOX inhibitory agent. According to this exemplary embodiment, the arNOX inhibitory agent is a naractin, the naractin being effective in decreasing the effects of aging. In some exemplary embodiments according to the invention, the naractin is extracted and/or purified from N. tazetta, willow, maize, crepis, poplar, viburnam, mold—especially Aspergillus, alangium, birch, bupleurum, colchicum, spurge, filipendulum, gardenia, lithospermum, tobacco or mistletoe.

In various other exemplary embodiments, the naractin is a salicylate or a derivative thereof. In some exemplary embodiments, the salicylate is salicylate is salicin, salicylic acid, salicyl hydroxamate, derivatives or combinations thereof. In various exemplary embodiments, the naractin is derived from Alangium chinense, A. platanifolium, A. premnifolium, Aspergillus niger, Betula alba, Bupleurum falcatum, Catharanthus roseus, Chosenia bracteosa, Colchicum autumnale, Crepis foetida, C. rhoeadifolia, Datura inoxia, Duboisia myoporoides, Eleutherococcus setchuensis, Euphorbia salicifolia, Filipendula ulmaria, Foeniculum vulgare, Gardenia jasminoides, Lithospermum erythrorhizon, Nicotiana tabacum, Populus alba, P. balsamifera, P. davidiana, P. deltoides, P. euphratica, P. grandidentata, P. heterophylla, P. lasiocarpa, P. maximowiczii, P. nigra, P. sieboldii, P. simonii, P. tacamahaca, P. tomentosa, P. tremula, P. tremuloides, P. trichocarpa, Salix acutifolia, S. alba, S. americana, S. arctica, S. aurita, S. babylonica, S. basfordiana, S. caesia, S. calodendron, S. capitata, S. caprea, S. chaenomeloides, S. cinerea, S. daphnoides, S. fragilis, S. geminata, S. gracilis, S. gracilistyla, S. gracilistyloides, S. gymnolepis, S. hastata, S. herbacea, S. incana, S. koriyanagi, S. lapponum, S. lasiandra, S. lasiolepis, S. matsudana, S. myrsinifolia, S. nigricans, S. orestera, S. pentandra, S. pentandroides, S. petiolaris, S. phylicifolia, S. purpurea, S. repens, S. rubra, S. schwerinii, S. scouleriana, S. smithiana, S. songarica, S. species, S. stipularis, S. tetrasperma, S. tremuloides, S. triandra, S. viminalis, Toisusu urbaniana, Viburnum henryi, V. prunifolium, V. rhytidophyllum or Viscum album.

In various other exemplary embodiments, the naractin is a salicylate or a derivative thereof. In some exemplary embodiments, the salicylate is salicylate is salicin, salicylic acid, salicyl hydroxamate, derivatives or combinations thereof

In various other exemplary embodiments, the composition further includes a cosmetically or pharmaceutically acceptable carrier. In some exemplary embodiments, the naractin inhibitory agent is present together with other arNOX inhibitors derived from naturally occurring sources including but not limited to broccoli, shitake, coleus rosemary, lotus, artichoke, sea rose tangerine, Oenothera biennis, astaxanthin, red orange, Schisandra chinensis, Lonicera, Fagopyrum, carrot or olive. In various exemplary embodiments, the naractin augments the effects of the additional arNOX inhibitory agents.

Those of skill in the art will recognize that the arNOX inhibitory compositions described herein can be administered in any convenient manner. In some exemplary embodiments such forms of administration include a cream, a milk, a lotion, a gel, a suspension of lipid or polymeric microspheres or nanospheres or vesicles, a soap, a shampoo or a sunscreen. In various exemplary embodiments, the effects of aging ameliorated by the instant invention include, but are not limited to lines, wrinkles, hyperpigmentation, dehydration, loss of elasticity, angioma, dryness, itching, telangietasias, actinic purpura, seborrheic keratoses, lack of hydration, decrease in collagen or actinic keratoses. In these and other exemplary embodiments, the invention, the arNOX inhibitory agent is provided at a concentration of between about 5 μg/ml to about 500 μg/ml.

In still other exemplary embodiments, the invention comprises a method to inhibit the generation of reactive oxygen species by aging-related isoform of NADH oxidase, to ameliorate the effects of aging comprising: administering a therapeutically effective amount of a composition comprising at least one of salicin, salicylic acid, salicyl hydroxamate to a patient in need thereof, such that generation of reactive oxygen species by aging-related isoform of NADH oxidase, is inhibited and wherein an effect of aging is ameliorated. In various exemplary embodiments he method further comprises an extract, or purified extract, from least one of broccoli, shitake, coleus rosemary, lotus, artichoke, sea rose tangerine, Oenothera biennis, astaxanthin, red orange, Schisandra chinensis, Lonicera, Fagopyrum, carrot, Narcissus tazetta, olive, willow, oat or maize. In various exemplary embodiments according to the invention the composition is applied as a cream, a milk, a lotion, a gel, a suspension of lipid or polymeric microspheres or nanospheres or vesicles, a soap, a shampoo or a sunscreen. In these exemplary embodiments, the effects of aging include, but are not limited to, lines, wrinkles, hyperpigmentation, dehydration, loss of elasticity, angioma, dryness, itching, telangietasias, actinic purpura, seborrheic keratoses, lack of hydration, decrease in collagen or actinic keratoses.

In still other exemplary embodiments, the invention includes a cosmetic method for ameliorating the effects of aging comprising applying to the skin a cosmetic composition comprising: an effective amount of a naractin sufficient to inhibit arNOX, wherein at least one arNOX mediated effect of aging is inhibited. In these embodiments, the naractin is a salicylate a salt or a derivative thereof. In various exemplary embodiments, the salicylate is salicin, salicyl hydroxamte, or salicylic acid. In various other embodiments of the method according to the invention, the cosmetic composition further includes a plant extract comprising: carrot extract, olive extract, broccoli extract, shitake extract, coleus, extract rosemary extract, lotus extract, artichoke extract, sea rose extract tangerine extract, Oenothera biennis extract, red orange extract, Schisandra chinensis extract, Lonicera extract, Fagopyrum extract, willow extract, maize, oat or Narcissus tazetta extract. In these exemplary embodiments, the naractin is provided together with a cosmetically acceptable carrier.

In various exemplary embodiments, the effects of aging ameliorated by the method according to the invention include lines, wrinkles, hyperpigmentation, dehydration, loss of elasticity, angioma, dryness, itching, telangietasias, actinic purpura, seborrheic keratoses, lack of hydration, decrease in collagen or actinic keratoses.

In various embodiments of the method according to the invention, the naractin is applied at least once a day. In some exemplary embodiments, the naractin is provided in a cosmetic preparation at a concentration of between about 5 μg/ml to about 500 μg/ml. In these exemplary embodiments, the cosmetic composition according to the invention is administered as a cream, a milk, a lotion, a gel, a suspension of lipid or polymeric microspheres or nanospheres or vesicles, a soap, a shampoo or a sunscreen.

In yet other embodiments according to the invention, the invention comprises a kit for applying a cosmetic useful in ameliorating the effects of aging comprising: at least one naractin; and instruction for use. In some exemplary embodiments the kit further comprises a cosmetic preparation suitable as a carrier for the at least one arNOX inhibitory plant extract.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be apparent from the description, as set forth hereinafter.

### BRIEF DESCRIPTION OF THE FIGURES

Various exemplary embodiments of the compositions and methods according to the invention will be described in detail, with reference to the following figures wherein:

FIG. 1a shows the activity pattern of arNOX isoforms from a saliva sample of a 72 year old (y/o) male (BL=base line) and partial inhibition after addition of an extract of N. tazetta powder (central panel). The right-hand panel shows the nearly complete inhibition of activity following addition to the N. tazetta powder of salicyl hydroxamate (SHAM) (Naractin 1) ingredient. FIG. 1b is the same as 1a except that instead of SHAM the inhibitory effect of the N. tazetta powder was augmented with salicin (Naractin 3). The Naractins form reddish complexes with iron with spectral properties similar to the reddish colors that characterize portions of N. tazetta bulb parts and extracts that contain arNOX-inhibitory substances as shown in FIG. 2.

FIGS. 2a-f illustrate regions of the N. tazetta bulb and extracts that contain substances inhibitory to arNOX activity. Inhibitory substances are absent from the parts of the bulbs and/or extracts that do not become red or pink in color. FIG. 2a is a drawing of a bulb divided into the region comprised of leaves and the stem region. FIG. 2b is a photograph showing that the red-colored compounds associated with arNOX inhibition are localized in the stem region. FIG. 2c show different regions of the bulb from which extracts were made and have from different regions have different levels of red color development as shown in FIGS. 2d, 2e and 2f. Color development is rapid. Bulbs of N. tazetta varieties where extracts do not inhibit arNOX activity also fail to develop the red color.

FIG. 3 illustrates a similar reddish color of the vasculature of maize steeles (the central water conducting tissues of the root) after addition of iron chloride. The substance resulting in the red color is a naturally-occurring hydroxamate.

FIG. 4 shows inhibition of arNOX activity of saliva (72 y/o M) in the base line (BL) panel and inhibition by addition of a homogenate prepared from maize steeles. Maize sheaths (the tissue surrounding the steeles) neither became colored upon addition of iron nor inhibited arNOX activity.

FIG. 5 shows examples from analyses by thin layer chromatography of methanol extracts of N. tazetta bulbs (B) compared to the commercial N. tazetta powder (A). The plates with blue (UV) background show ultraviolet fluorescence. The plates with light background (BB) were stained with Berlin blue. The TLC system was dichloromethane:methanol:NH₄OH (10:1:0.2). In the laboratory-extracted sample, a reddish component just above but clearly separated from the material at the origin and suggestive of the presence of hydroxamates was much more evident in the laboratory-extracted sample than in the commercial powder. This was evident for both methanol (FIG. 5a) and water (FIG. 5b) extracts. The putative hydroxamate was seen also in the commercial powder but at much lower levels than in the laboratory-extracted sample once again correlating with levels of arNOX inhibitory activity.

FIG. 6 shows a spectral analyses of N. tazetta bulb and maize steele and sheath extracts compared to a known hydroxamate (SHAM) following addition of ferric chloride to form red-colored ferric hydroxamates. arNOX inhibitory-extracts of N. tazetta bulbs were red and exhibited an absorbance maximum around 550 nm. Extracts of N. pseudo narcissus bulbs which lacked inhibitory activity were colorless and showed no absorbance at 550 nm. Maize root steele (active) and maize root sheath (inactive) fractions were largely uncolored in the absence of added ferric chloride (−FeCl₂). However, in the presence of ferric chloride (+FeCl₂), red color appeared in the maize steele extracts with an absorbance maximum at about 550 nm whereas there was no absorbance maximum at 550 nm for the maize sheaths. Salicyl hydroxamate (SHAM), a commercially available stable hydroxamate also gave a red color with a maximum at about 550 nm. The negative control was uncolored and showed no absorbance at 550 nm.

FIG. 7 is a graph of the arNOX activity of ferricytochrome c as a function of SHAM dilution (log). The graph illustrates the dose-dependent inhibition of arNOX activity of saliva (72 y/o M) by a commercially available preparation of SHAM (Sigma-Aldrich, St. Louis, Mo.). Inhibition is largely complete at 11 mg/ml.

FIG. 8 is a graph of the arNOX activity of ferricytochrome c as a function of salicylic acid dilution (log). The graph illustrates the dose-dependent inhibition of arNOX activity of saliva (72 y/o M) as a function of salicylic acid concentration. Salicylic acid at a concentration of 1 mg/ml inhibits about 50%.

FIG. 9 is a graph of the arNOX activity of ferricytochrome c as a function of salicin dilution (log). The graph illustrates the dose-dependent inhibition of arNOX activity of saliva (72 y/o M) as a function of salicin concentration. Maximum inhibition of arNOX activity is achieved at a concentration of 1 mg/ml.

FIGS. 10a and 10b are graphs showing the arNOX inhibition of various “Naractin” combinations. FIG. 10a illustrates salicin augmentation of arNOX inhibition of a mixture of 4 mg/ml Schizandra powder plus N. tazetta extract (20 μl) in the presence of 1 mg/ml salicin. FIG. 10b illustrates the arNOX inhibition by a mixture of 4 mg/ml Schizandra powder plus 1 mg/ml N. tazetta powder in the presence of 1 mg/ml salicin. Of the mixture, 60 μl was added to at total of three ml of assay mixture containing saliva (72 y/o M) as the arNOX activity source.

### DETAILED DESCRIPTION OF THE EXEMPLARY EMBODIMENTS

The invention relates to agents for sequestering serum aging factors, and methods for using the same. More particularly, the invention relates to agents termed herein “Naractin” to denote any one of several naturally-occurring arNOX inhibitors either present in N. tazetta powder or capable of augmenting N. tazetta powder to an inhibitory level comparable to that of the fresh N. tazetta extracts, and to methods for using “Naractins” to prevent or treat disorders and complications of disorders resulting from cell damage caused by an aging-related isoform of NADH oxidase (arNOX). In one exemplary embodiment the agents of the invention comprise at least one naturally occurring Naractin. Such naturally occurring naractins are also capable of augmenting the anti-arNOX effect of other naturally occurring arNOX inhibitory agents.

The term “Naractin” is used herein to denote any one of several naturally-occurring arNOX inhibitors either present in N. tazetta powder (commercially available from, for example, Xian Aojing Science and Technology Developing CO., LTD, Xi'an, Shaanxi, CN) or capable of augmenting N. tazetta powder to an inhibitory level comparable to that of the fresh N. tazetta extracts. The inventors have currently identified three known substances, chemically pure, with “Naractin” activity. It should further be noted that, though the “Naractins” have been herein identified from N. tazetta extract they may be present from other natural sources such as, for example, willow, maize and oat and, of course, the invention encompasses Naractins regardless of their source.

As used herein the term “cosmetic” refers to a substance intended to be applied to the body for cleansing, beautifying, promoting attractiveness, or altering the appearance.

As used herein the term “extract” refers to a solution obtained by steeping or soaking a substance in a solvent and removing the active ingredient. The solvent can be any suitable solvent including but not limited to alcohol, water or the like. In some instances the extract is concentrated or the solvent can be evaporated and the active ingredient resuspended or solubilized in a different solvent. As described herein, the N. tazetta extract is made by separating the stem region from the bulb and homogenizing one stem region in 3 ml of distilled water.

As used herein, the term “disorder” refers to any condition of a living animal or plant body or of one of its parts that impairs normal functioning comprising any ailment, disease, illness, clinical condition, pathological condition, weakened condition, unsound condition, and any abnormal or undesirable physical condition.

As used herein, the term “reactive oxygen species” refers to oxygen derivatives from oxygen metabolism or the transfer of free electrons, resulting in the formation of free radicals (e.g., superoxides or hydroxyl radicals).

As used herein, the term “antioxidant” refers to compounds that neutralize the activity of reactive oxygen species or inhibit the cellular damage done by said reactive species.

As used herein, the term “pharmaceutically acceptable carrier” refers to a carrier medium that does not interfere with the effectiveness of the biological activity of the active ingredient, is chemically inert, and is not toxic to the patient to whom it is administered.

As used herein, the term “pharmaceutically acceptable derivative” refers to any homolog, analog, or fragment corresponding to the formulations described in this application, which exhibit antioxidant activity, and is relatively non-toxic to the subject.

The term “therapeutic agent” refers to any molecule, compound, or treatment, preferably an antioxidant, which assists in the prevention or treatment of the disorders, or complications of disorders caused by reactive oxygen species.

The term “agent that sequesters arNOX” refers to any molecule, compound, or treatment that interacts with arNOX, thus decreasing the reaction of arNOX with other substrates and inhibits the ability of arNOX to generate reactive oxygen species.

There term “salicylates” as used herein refers to salts, conjugates or derivatives of salicin whether derived from naturally occurring sources or synthesized de novo. Such salicylates include, for example, salicylic acid, salicin hydroxamate and salicin itself, their derivatives, salts and conjugates.

During the course of their research, the inventors had found that certain commercial preparations of N. tazetta extract contained active agents having anti-arNOX activity. The present invention was prompted by the observation that some samples of IBR-Dormin® N. tazetta bulb extract (Israeli Biotechnology Research, Ltd., Israel) had virtually no inhibitory activity toward arNOX whereas other lots from the same vendor were strongly inhibitory. The inventors hypothesized that either the arNOX inhibitor was unstable in the absence of special preservatives or that the arNOX inhibitor had been lost during N. tazetta extract purification. It should be noted, however, that the commercially available N. tazetta powder available under the trade name IBR-Dormin® is especially processed with the N. tazetta bulbs are in dormancy. The theory being that at the growth stage the bulbs produce quantities of unidentified compounds referred to as “dormins”. Such “dormins” are further hypothesized to elicit a state of dormancy in cells or tissues. Thus, from the instantly disclosed data it is shown that the N. tazetta “dormin” is not the same agent as the herein identified as “Naractins.”

To test these possibilities, the inventors obtained a small number of paper white (N. tazetta) bulbs. Water and ethanolic extracts inhibited arNOX. Comparisons were made with N. pseudo-narcissus (daffodil) and N. jonquilla (jonquil) both of which were largely without activity. Activity was correlated with a red coloration enhanced by addition of ferric iron, a characteristic of naturally-occurring hydroxamates as are found in corn and oat seedlings data not shown) which also inhibit arNOX. Hydroxamates have a chemistry which might be expected to be conducive to arNOX inhibition. The most readily available hydroxamate for evaluation was salicyl hydroxamate (Naractin 1), also known as SHAM, a known inhibitor of the alternate oxidase of plants. SHAM was tested and was found to augment the arNOX inhibitory response of N. tazetta powder. A level of inhibition of greater than 90% was obtained with N. tazetta powder augmented with low levels of SHAM. While there were insufficient N. tazetta bulbs to complete the characterization of the active component but the work was continued with N. tazetta powder (extract) (Xi'an Aojing Science and Technology Developing Co., LTD., Xi'an, Shaanxi, China; 20:1 available in both bulb and flower extract). N. tazetta powder does appear to contain low levels of a naturally-occurring hydroxamate but at levels much less than extracts prepared from N. tazetta bulbs.

A question posed was whether the hydroxamate or the salicylic acid portion was most important for arNOX inhibitor activity. The results suggest that both are important. Salicylic acid (Naractin 2) of itself is a very potent arNOX inhibitor which also gives a red color when reacted with ferric iron. Esters of salicylic acid or aspirin do not inhibit arNOX. An aqueous extract of willow bark, a natural source of salicylates, was tested and also found to inhibit arNOX. The major salicylate of willow bark, salicin, a glycoside, also was effective as an inhibitor (Naractin 3).

Therefore, in one exemplary embodiment the invention includes a topical composition useful for ameliorating the effects of aging comprising an effective amount of at least one arNOX inhibitory agent. According to this exemplary embodiment, the arNOX inhibitory agent is a naractin, the naractin being effective in decreasing the effects of aging. In some exemplary embodiments according to the invention, the naractin is purified from N. tazetta, willow, maize, crepis, poplar, viburnam, mold—especially Aspergillus, alangium, birch, bupleurum, colchicum, spurge, filipendulum, gardenia, lithospermum, tobacco or mistletoe.

In various other exemplary embodiments, the naractin is a salicylate or a derivative thereof. In some exemplary embodiments, the salicylate is salicylate is salicin, salicylic acid, salicyl hydroxamate, derivatives or combinations thereof. In various exemplary embodiments, the naractin is derived from Alangium chinense, A. platanifolium, A. premnifolium, Aspergillus niger, Betula alba, Bupleurum falcatum, Catharanthus roseus, Chosenia bracteosa, Colchicum autumnale, Crepis foetida, C. rhoeadifolia, Datura inoxia, Duboisia myoporoides, Eleutherococcus setchuensis, Euphorbia salicifolia, Filipendula ulmaria, Foeniculum vulgare, Gardenia jasminoides, Lithospermum erythrorhizon, Nicotiana tabacum, Populus alba, P. balsamifera, P. davidiana, P. deltoides, P. euphratica, P. grandidentata, P. heterophylla, P. lasiocarpa, P. maximowiczii, P. nigra, P. sieboldii, P. simonii, P. tacamahaca, P. tomentosa, P. tremula, P. tremuloides, P. trichocarpa, Salix acutifolia, S. alba, S. americana, S. arctica, S. aurita, S. babylonica, S. basfordiana, S. caesia, S. calodendron, S. capitata, S. caprea, S. chaenomeloides, S. cinerea, S. daphnoides, S. fragilis, S. geminata, S. gracilis, S. gracilistyla, S. gracilistyloides, S. gymnolepis, S. hastata, S. herbacea, S. incana, S. korinyanagi, S. lapponum, S. lasiandra, S. lasiolepis, S. matsudana, S. myrsinifolia, S. nigricans, S. orestera, S. pentandra, S. pentandroides, S. petiolaris, S. phylicifolia, S. purpurea, S. repens, S. rubra, S. schwerinii, S. scouleriana, S. smithiana, S. songarica, S. species, S. stipularis, S. tetrasperma, S. tremuloides, S. triandra, S. viminalis, Toisusu urbaniana, Viburnum henryi, V. prunifolium, V. rhytidophyllum, or Viscum album.

In various other exemplary embodiments, the naractin is a salicylate or a derivative thereof. In some exemplary embodiments, the salicylate is salicylate is salicin, salicylic acid, salicyl hydroxamate, derivatives or combinations thereof

In various other exemplary embodiments, the composition further includes a cosmetically or pharmaceutically acceptable carrier. In some exemplary embodiments, the naractin inhibitory agent is present together with other arNOX inhibitors derived from naturally occurring sources including but not limited to broccoli, shitake, coleus rosemary, lotus, artichoke, sea rose tangerine, Oenothera biennis, astaxanthin, red orange, Schisandra chinensis, Lonicera, Fagopyrum, carrot, or olive. In various exemplary embodiments, the naractin augments the effects of the additional arNOX inhibitory agents.

Those of skill in the art will recognize that the arNOX inhibitory compositions described herein can be administered in any convenient manner. In some exemplary embodiments such forms of administration include a cream, a milk, a lotion, a gel, a suspension of lipid or polymeric microspheres or nanospheres or vesicles, a soap, a shampoo or a sunscreen. In various exemplary embodiments, the effects of aging ameliorated by the instant invention include, but are not limited to lines, wrinkles, hyperpigmentation, dehydration, loss of elasticity, angioma, dryness, itching, telangietasias, actinic purpura, seborrheic keratoses, lack of hydration, decrease in collagen or actinic keratoses. In these and other exemplary embodiments, the invention, the arNOX inhibitory agent is provided at a concentration of between about 5 μg/ml to about 500 μg/ml.

In still other exemplary embodiments, the invention comprises a method to inhibit the generation of reactive oxygen species by aging-related isoform of NADH oxidase, to ameliorate the effects of aging comprising: administering a therapeutically effective amount of a composition comprising at least one of salicin, salicylic acid, salicyl hydroxamate to a patient in need thereof, such that generation of reactive oxygen species by aging-related isoform of NADH oxidase, is inhibited and wherein an effect of aging is ameliorated. In various exemplary embodiments the method further comprises an extract, or purified extract, from least one of broccoli, shitake, coleus rosemary, lotus, artichoke, sea rose tangerine, Oenothera biennis, astaxanthin, red orange, Schisandra chinensis, Lonicera, Fagopyrum, carrot, Narcissus tazetta, olive, willow, oat, maize, crepis, poplar, viburnam, mold—especially Aspergillus, alangium, birch, bupleurum, colchicum, spurge, filipendulum, gardenia, lithospermum, tobacco or mistletoe. In various exemplary embodiments according to the invention the composition is applied as a cream, a milk, a lotion, a gel, a suspension of lipid or polymeric microspheres or nanospheres or vesicles, a soap, a shampoo, an elixir or a sunscreen. In these exemplary embodiments, the effects of aging include, but are not limited to, lines, wrinkles, hyperpigmentation, dehydration, loss of elasticity, angioma, dryness, itching, telangietasias, actinic purpura, seborrheic keratoses, lack of hydration, decrease in collagen or actinic keratoses.

In still other exemplary embodiments, the invention includes A cosmetic method for ameliorating the effects of aging comprising applying to the skin a cosmetic composition comprising: an effective amount of a naractin sufficient to inhibit arNOX, wherein at least one arNOX mediated effect of aging is inhibited. In these embodiments, the naractin is a salicylate a salt or a derivative thereof. In various exemplary embodiments, the salicylate is salicin, salicyl hydroxamte, or salicylic acid. In various other embodiments of the method according to the invention, the cosmetic composition further includes a plant extract comprising: carrot extract, olive extract, broccoli extract, shitake extract, coleus, extract rosemary extract, lotus extract, artichoke extract, sea rose extract tangerine extract, Oenothera biennis extract, red orange extract, Schisandra chinensis extract, Lonicera extract, Fagopyrum extract, willow extract, maize, crepis, poplar, viburnam, mold—especially Aspergillus, alangium, birch, bupleurum, colchicum, spurge, filipendulum, gardenia, lithospermum, tobacco, mistletoe, oat or Narcissus tazetta extract. In these exemplary embodiments, the naractin is provided together with a cosmetically acceptable carrier.

In various exemplary embodiments, the effects of aging ameliorated by the method according to the invention include lines, wrinkles, hyperpigmentation, dehydration, loss of elasticity, angioma, dryness, itching, telangietasias, actinic purpura, seborrheic keratoses, lack of hydration, decrease in collagen or actinic keratoses.

In various embodiments of the method according to the invention, the naractin is applied at least once a day. In some exemplary embodiments, the naractin is provided in a cosmetic preparation at a concentration of between about 5 μg/ml to about 500 μg/ml. In these exemplary embodiments, the cosmetic composition according to the invention is administered as a cream, a milk, a lotion, a gel, an elixir, a suspension of lipid or polymeric microspheres or nanospheres or vesicles, a soap, a shampoo or a sunscreen.

In yet other embodiments according to the invention, the invention comprises a kit for applying a cosmetic useful in ameliorating the effects of aging comprising: at least one naractin; and instruction for use. In some exemplary embodiments the kit further comprises a cosmetic preparation suitable as a carrier for the at least one arNOX inhibitory plant extract.

The antioxidants, cellular components, and target proteins defined herein are abbreviated as follows:

| | |
|---|---|
| mitochondrial DNA | mtDNA |
| nicotinamide adenine dinucleotide | NADH |
| cell surface hydroquinone (NADH) oxidase with | NOX |
| protein disulfide-thiol isomerase activity | |
| NOX specific to non-cancer cells | cNOX |
| NOX specific to aged cells | arNOX |
| NOX specific to cancer cells | tNOX |
| low density lipoprotein | LDL |
| plasma membrane oxido-reductase chain | PMOR |
| ubiquinone or coenzyme Q | CoQ |
| coenzyme Q₁₀ | CoQ₁₀ |
| reactive oxygen species | ROS |

The Invention

The present invention is directed to naturally occurring agents identified and purified from the Narcissus tazetta bulb and maize and may be administered either internally or topically. These agents specifically inhibit arNOX and ameliorate some of its aging related effects. Such agents can take the form of isolated agents or plant extracts. Further, while arNOX inhibitory agents can be used alone, they may also be used as compositions comprising multiple arNOX inhibitory agents and/or formulations including compounds having other beneficial effects on the body. In particular, the inventors have found that by adding arNOX inhibitors to cosmetics, the inhibitors can have beneficial effects that augment the normal skin care regimen.

In yet another exemplary embodiment, the invention comprises a cosmetic composition for ameliorating the effects of aging comprising a cosmetically effective amount of at least one arNOX inhibitory agent wherein the arNOX inhibitory agent is effective in decreasing the effects of aging upon the skin. In one version of this exemplary embodiment, the invention includes a cosmetically acceptable carrier. In this embodiment, the carrier may include powders, emollients, lotions, creams, liquids and the like. In some exemplary embodiments, the arNOX inhibitory agent is derived from a plant. In particular exemplary embodiments, the plant is selected from broccoli, shitake, coleus, rosemary, lotus, artichoke, sea rose, tangerine, Oenothera biennis, astaxanthin, red orange, Schisandra chinensis, Lonicera, Fagopyrum, carrot, Narcissus tazetta, willow, maize, maize, crepis, poplar, viburnam, mold—especially Aspergillus, alangium, birch, bupleurum, colchicum, spurge, filipendulum, gardenia, lithospermum, tobacco, mistletoe, oat or olive.

It should be appreciated that the cosmetic composition according to this exemplary embodiment can be administered in any exemplary manner. For example, in some exemplary embodiments, the cosmetic composition according to the invention is applied topically, orally, parenterally, transdermally or rectally. In some exemplary embodiments, the composition is formulated as a cream, a milk, a lotion, a gel, a suspension of lipid or polymeric microspheres or nanospheres or vesicles, a soap or a shampoo.

In still other exemplary embodiments, the invention includes a cosmetic method for ameliorating the effects of aging comprising applying to the skin a cosmetic composition comprising an effective amount of an arNOX inhibitor, wherein at least one arNOX mediated effect of aging is inhibited. In some exemplary embodiments according to the invention, the arNOX inhibitor is a plant extract. In other exemplary embodiments, the arNOX inhibitor is purified from a plant extract. In various exemplary embodiments according to the invention the arNOX inhibitory agent is present in a concentration of between about 5 μg/ml to about 500 μg/ml. In various exemplary embodiments, the concentration of the active agent is present in a concentration of between about 15 to 100 μg/ml. In some exemplary embodiments, the cosmetic composition according to the invention is applied topically, orally, parenterally, transdermally, rectally or by any effective method. In some exemplary embodiments, the composition is formulated as a cream, a milk, a lotion, a gel, a suspension of lipid or polymeric microspheres or nanospheres or vesicles, a soap or a shampoo.

In still other exemplary embodiments, the invention comprises a kit. In this embodiment, the kit may include a volume of an arNOX inhibitory agent and instruction for use. In various exemplary embodiments, the kit may further include a cosmetic preparation such that the arNOX inhibitory agent can be added to the cosmetic preparation prior to use.

It should be appreciated that while in some exemplary embodiments of the invention, one arNOX inhibitory agent is used, in other exemplary embodiments more than one extract or arNOX inhibitory agent are used together. Further, it should be appreciated that in various exemplary embodiments of the invention, the one or various arNOX inhibitory agents may be applied or administered in various ways. Such as, for example, topical administration, in the form of a cream, a milk, a lotion, a gel, a suspension of lipid or polymeric microspheres or nanospheres or vesicles, a soap, a shampoo or a sunscreen and in the form of a tea or capsule or any other effective manner.

Plasma Membrane Hydroquinone (NADH) Oxidase (NOX):

The plasma membrane NADH oxidase (NOX) is a unique cell surface protein with hydroquinone (NADH) oxidase and protein disulfide-thiol interchange activities that normally responds to hormone and growth factors. A hormone insensitive and drug-responsive form of the activity designated tNOX also has been described, which is specific for cancer cells. Evidence exists that NOX proteins, under certain conditions, are capable of the production of ROS. For example, ultraviolet light as a source of oxidative stress in cultured cells is used to initiate superoxide generation (Morré et al., 1999, Biofactors 9:179-187) (See U.S. Pat. No. 5,605,810, which is incorporated herein by reference in its entirety).

Isolation and Characterization of arNOX inhibitors from N. tazetta

The investigative series leading to the invention was prompted by observation certain samples of a commercially available N. tazetta extract, (commercially available as IBR-Dormin®, IBR, Inc, Israel) were often strongly inhibitory to arNOX whereas other samples y of different lots but the same type of preparation were not. The inventors hypothesized that either the arNOX inhibition was due to something unstable in the absence of special preservatives or that the arNOX inhibitory constituents of the active preparations had been lost from the inactive preparations during extract purification. It should be noted that recently a product known as IBR-Dormin® has been described, which relates to anti-proliferative agents derived from plants. See, U.S. patent application publication No. 20060160702, application Ser. No. 11/289,156 (the '156 application) to Soudant et al. According to the '156 application, anti-proliferative agents are found in plants that are capable of inducing a plant organ into a state of dormancy (Abstract). Thus, the anti-proliferative agents coined “dormins” are suggested to function by slowing down aging via slowing down cell proliferation. The “dormins” are harvested by making extracts of the plants which have entered their dormant stage. Thus, only extract of dormant plants can be used as “dormins” for this property.

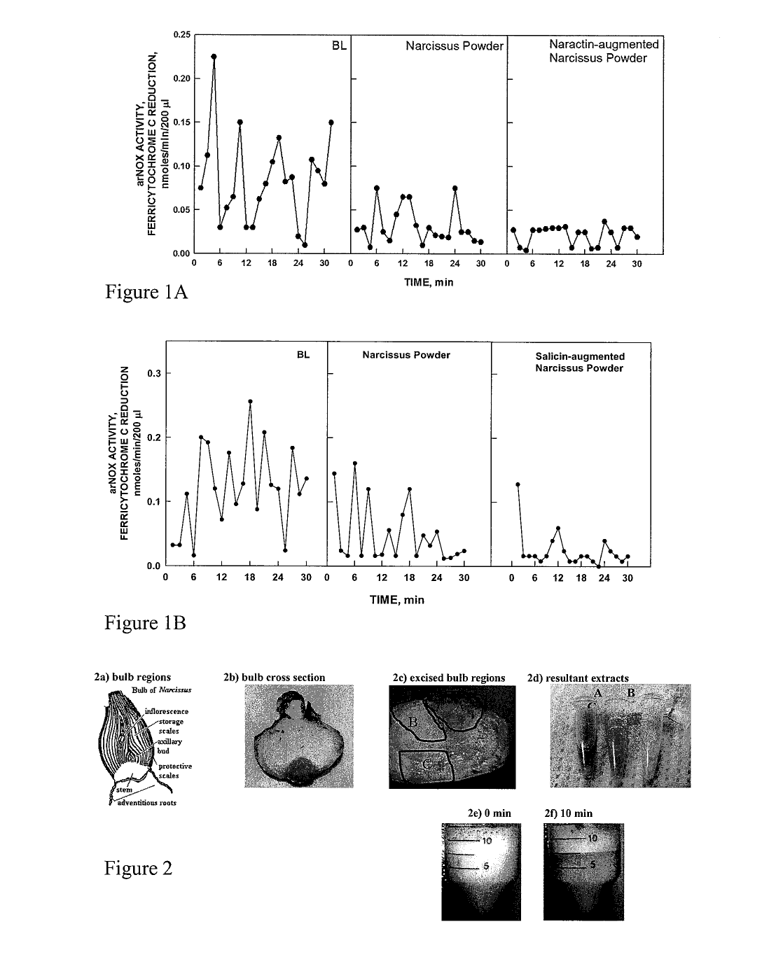

Because the inventors noticed that different N. tazetta extracts of the commercially available N. tazetta powder of both bulb and flower (Xian Aojing Science and Technology Developing CO., LTD, Xian, CN) had varying degrees of arNOX inhibition, the inventors conducted a series of experiments to identify the arNOX inhibitory substance. To test the above possibilities, bulbs of paper white (N. tazetta) narcissus were obtained from a commercial garden provider (Brent and Becky's bulbs, Gloucester, Va.). Comparisons were made with N. pseudo-narcissus (daffodil) and N. jonquilla (jonquil) both of which were largely without activity. Activity was correlated with a red coloration enhanced by addition of ferric iron a characteristic of naturally occurring hydroxamates. The most readily available hydroxamate for evaluation was salicyl hydroxamate (SHAM) (Naractin 1). Water and ethanolic extracts were prepared which inhibited arNOX and augmented the inhibition when combined with N. tazetta powder from the flower of low activity (e.g., FIG. 1a). FIG. 1b is similar to 1a showing augmentation of N. tazetta powder derived from the bulb (Xian Aojing, LTD) extracted with water and augmented using salicin (Sigma-Aldrich, St. Louis, Mo.) (Naractin 3). Comparisons were made with similar extracts of Narcissus pseudo-narcissus (daffodil) and Narcissus jonquilla (jonquil) extracts which lacked arNOX inhibitory activity entirely.

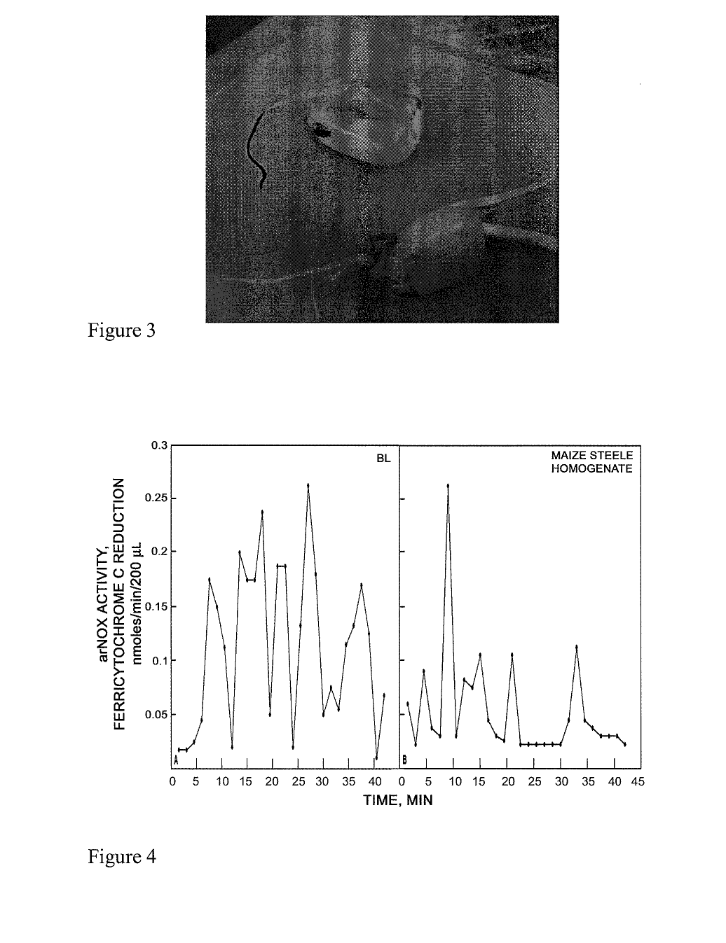

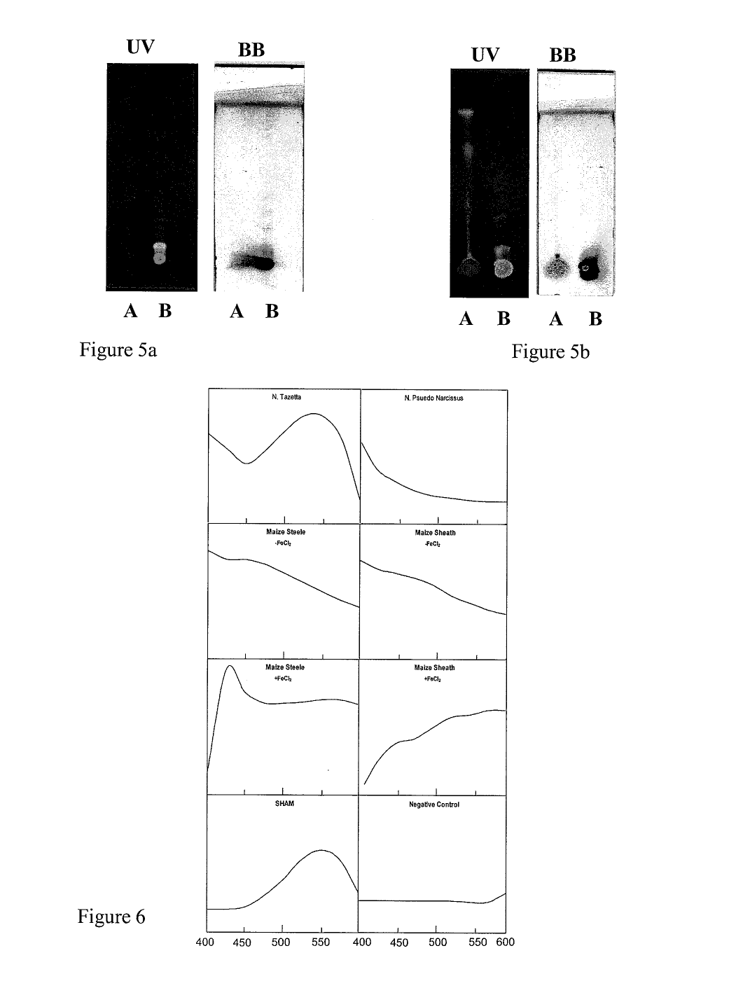

As illustrated in FIG. 2, activity of the N. tazetta extracts was correlated with a red coloration enhanced by the addition of ferric iron, a characteristic of naturally-occurring hydroxamates, such as those found in corn (maize) (FIG. 3) and oat (not shown) seedlings which also inhibit arNOX (FIG. 4). Further evidence for the potential ability of the active components of N. tazetta extracts to comprise naturally-occurring hydroxamates was provided by thin layer chromatographic analyses (FIG. 5), from spectral studies of their iron adducts, from the steeles (the central vasculature of the maize root where the hydroxamates are concentrated corresponding to the potentially hydroxamate-rich stem vasculature of the N. tazetta bulb illustrated in FIG. 2) of maize roots (FIG. 6) and by comparison to a commercially available hydroxamate (salicyl hydroxamate (SHAM) commercially available from, for example Sigma-Aldrich, St Louis, Mo.)

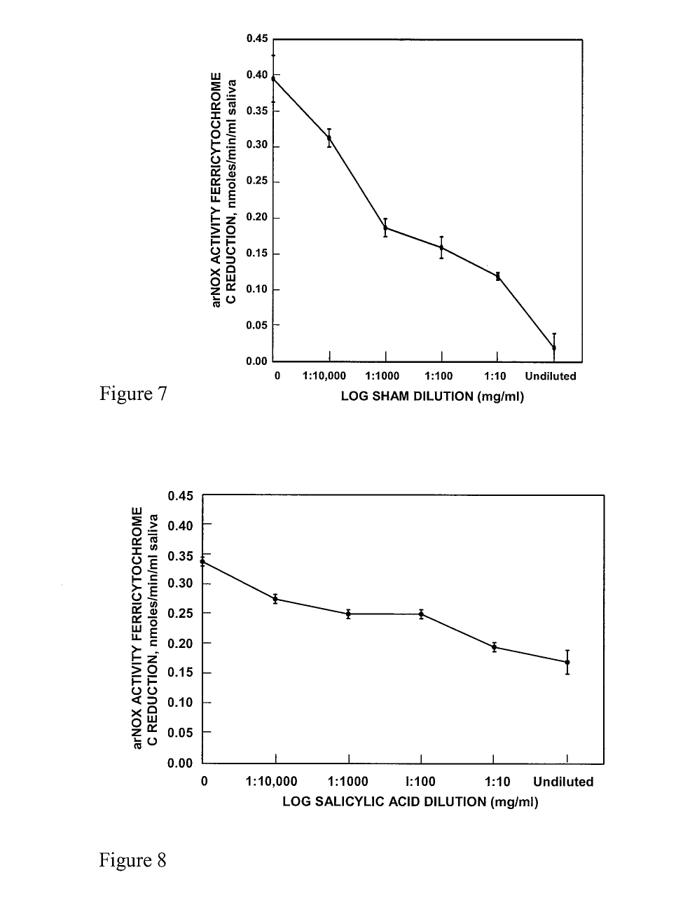

SHAM, was tested for inhibition of arNOX activity and greater than 90% inhibition of arNOX activity of saliva from a 72 y/o male was obtained at a concentration of 1 mg/ml (FIG. 7). SHAM was also found to augment the inhibition of arNOX activity of partially active N. tazetta powders (FIG. 1). The commercially available N. tazetta powder does appear to contain low levels of a naturally-occurring hydroxamate possibly similar to SHAM but at levels much less than that found in extracts prepared from fresh N. tazetta bulbs (FIGS. 5a and 5b).

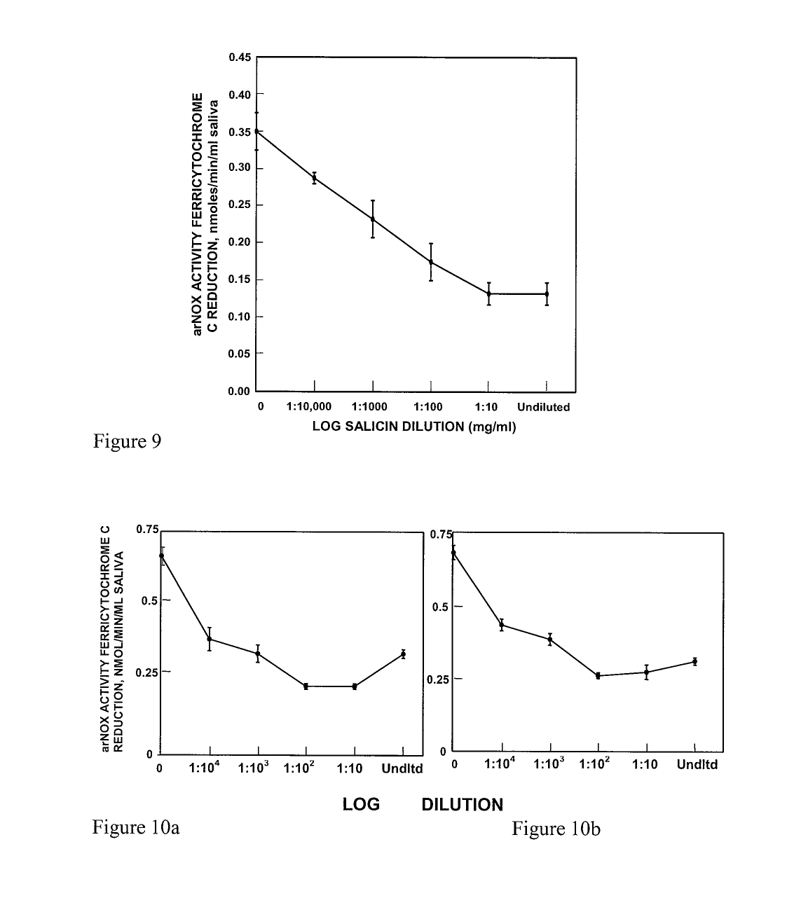

To answer the question of whether or not the salicylic portion of the stabilized salicyl hydroxamate was important to inhibition of arNOX activity, salicylic acid (Naractin 2, commercially available from, for example, Sigma-Aldrich, St Louis, Mo. was tested as an inhibitor of arNOX activity of saliva (72 y/o M) and was found to inhibit arNOX (FIG. 8). Salicylic acid also gave a red color when reacted with ferric iron similar to that given by the hydroxamates. Esters of salicylic acid and/or aspirin did not inhibit arNOX. An aqueous extract of willow bark, a natural source of salicylates, was tested and found to inhibit arNOX as well. Salicin (Sigma-Aldrich, St Louis, Mo.) (designated herein as Naractin 3), the major salicylate of willow bark, was subsequently tested and found to be active at a ten-fold lower concentration than either SHAM (Naractin 1) or salicylic acid (Naractin 2) (FIG. 9).

Not only was salicin active as a single agent (FIG. 9), it was also active in augmenting the arNOX inhibition of mixtures of other arNOX inhibiting agents from natural sources such as from Schizandra chinensis powder and from N. tazetta powder (FIGS. 10a and 10b). Of the three chemically pure Naractins (a term used to denote any one of several naturally occurring arNOX inhibitors either present in N. tazetta powder or capable of augmenting N. tazetta powder to an inhibitory level comparable to that of the most active fresh N. tazetta extracts) identified and tested, salicin appeared to be the most promising. Further, salicin is stable, water soluble, non-irritating and relatively inexpensive.

Generally, the characteristics of aged cells includes those that express and/or shed arNOX, and include, but are not limited to, those exhibiting one or more of the following characteristics: an age-related PMOR system, the ability to generate reactive oxygen species, and have functionally defective mitochondria. One embodiment of the invention is the utilization of agents to reduce the negative effects of aging cells.

The symptoms of aging skin include dryness, itchiness, thinning or thickening of the skin, wrinkles and fine lines, areas of hyperpigmentation (called age or liver spots), and a mottled appearance. Aging skin has been shown to have a decrease in collagen and a concomitant decrease in elasticity. In addition, aging skin has increased amounts of cleaved collagen and cross-linked proteins. Superoxide radicals have been indicated in these processes. The skin may take more time to heal when injured. Blood vessels are easier to see through the thinning skin, also because they become dilated with age. These blood vessels may be visible as red dome-like formations on the skin (cherry angiomas), or as broken capillaries on the face (telangietasias). Many people develop senile or actinic purpura, which are purplish spots or patches on the skin created by small hemorrhages in the skin. Older skin has less protection against sun damage because protective cells called melanocytes decrease with age. Aging skin is also more likely to develop a variety of benign and pre-cancerous growths, such as seborrheic and actinic keratoses. Seborrheic keratoses often have a rough, brown appearance, and look like a wart. They are benign. Actinic keratoses are small, scaly growths on areas of the skin that have received sun exposure. They are an early sign of skin cancer

The invention encompasses the use of topical administration of natural plant extracts, alone or in the form of a cream emollient, lotion, gel, emollient or the like, to maintain skin vitality. A preferred embodiment of the invention comprises the topical administration of a cream, which comprises an arNOX inhibiting extract, to the skin of patients to maintain and improve skin vitality.

Cosmetic Treatment of Skin

The present invention provides compositions comprising active agent(s), which prevent and/or ameliorates skin damage and associated conditions, particularly those resulting from aging and associated with arNOX. Further, the invention encompasses methods for utilizing said compositions. The stratum corneum is the layer of the skin that forms the top surface layer and serves to protect the skin while controlling moisture and the flow of substances in and out of the skin. As this barrier function is broken down, the skin suffers damaging effects, thus further contributing to premature aging. These damaging effects causing premature aging of the skin are a concern for many individuals wishing to maintain healthy, youthful looking and feeling skin. Reactive oxygen species participate in a number of destructive reactions potentially lethal to cells. Reactive oxygen species are responsible in part for deleterious cellular interactions including impairing fibroblast cells ability to produce healthy collagen and elastin. Furthermore, the skin is subject to deterioration through dermatological disorders, environmental abuse (wind, air conditioning, central heating) or through the normal aging process (chronoaging), which may be accelerated by exposure of skin to sun (photoaging).

A preferred embodiment of the invention provides naturally occurring active agents from plants for the treatment of arNOX related damage to tissue, especially skin. The active agents prevent and/or ameliorate skin damage and associated conditions. In one embodiment of the invention the processed plant products sequester arNOX activity. In another embodiment of the invention, the processed plant products inhibit reactive oxygen species. In another embodiment agents and methods of the invention prevent and/or improve the health of the skin. For example, the agents may improve, tautness of skin, color and appearance of pores, elasticity, hydration and/or help diminish the appearance of fine lines and visible signs of aging. In another exemplary embodiment of the invention, the agents positively affect the body's natural production of collagen and elastin. In another embodiment, the agents of the invention minimize the effects of environmental agitators such as pollution, sun, free radicals and stress.

One embodiment of the invention provides compositions, and methods for using the same, for preventing and/or ameliorating dermatological disorders and the effects thereof.

One embodiment of the invention provides a composition for preventing and reducing the effects of the production of reactive oxygen species and methods for using the same. For example, the invention encompasses the use of active agents derived from plants to at least partially sequester or inhibit arNOX activity. Further, the invention contemplates the use of other synthetic and natural compounds to sequester arNOX activity.

The present invention discloses compositions, which treat the skin and delay the visible signs of actual aging and weathered skin such as wrinkles, lines, sagging, hyperpigmentation and age spots. The present invention also decreases the appearance and condition of sensitive, dry and/or flaky skin, serves to soothe red, and/or irritated skin, and treats spots, pimples, blemishes, and other skin irregularities.

The invention provides pharmaceutical or cosmetic compositions, methods of use, and pharmaceutical or cosmetic kits for the treatment of disorders resulting from oxidative changes in cells that result in aging by targeting an aging-related isoform of NADH oxidase (arNOX), shed into the sera by aging cells. The compositions may contain agents extracted from plants. For example, the compositions of the invention may comprise at least one extract shown to inhibit arNOX activity, whether alone or with other inhibition agents and, at least partially, inhibit or block the activity of an aging-related isoform of NADH oxidase shed into the sera by aging cells. The composition may comprise ubiquinones, natural extracts or agents derived therefrom known to comprise active agents useful in inhibiting arNOX, together with other compounds known in the art to make creams, lotions, emollients, gels and the like. Such other compounds may comprise gums, fillers, preservatives and the like.

In one embodiment a portion of, or all of these ingredients may be combined with other ingredients commonly found in anti-aging and repair serum formulations. Vehicles, other than, or in addition to water can include liquid or solid emollients, solvents, humectants, thickeners and powders. The vehicle may be from 0.1% to 99.9%, preferably from 25% to 80% by weight of the composition, and can, in the absence of other cosmetic adjuncts, form the balance of the composition. In one embodiment, the vehicle is at least 80% water, by weight of the vehicle. In another embodiment water comprises at between about 50% to 85% of the composition by weight. In yet another embodiment, water is present between about 0.1% to 55%, by weight of the composition. In other embodiments other vehicles are used in the above recited concentrations.

An oil or oily material may be present, together with an emulsifier to provide either a water-in-oil emulsion or an oil-in-water emulsion, depending largely on the average hydrophilic-lipophilic balance (HLB) of the emulsifier employed.

The inventive compositions may also include sunscreens. Sunscreens include those materials commonly employed to block ultraviolet light. Illustrative compounds are the derivatives of PABA, cinnamate and salicylate. For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the emulsions can vary depending upon the degree of protection desired from the sun's UV radiation.

Emollients may further be incorporated into cosmetic compositions of the present invention. Levels of such emollients may range from 0.5% to 50%, preferably between 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols, which may serve as emollients are linear and branched chain alkyl polyhydroxyl compounds. For example, propylene glycol, sorbitol and glycerin are preferred. Also useful may be polymeric polyols such as poly-propylene glycol and polyethylene glycol. Butylene and propylene glycol are also especially preferred as penetration enhancers.

Exemplary hydrocarbons which may serve as emollients are those having hydrocarbon chains anywhere from 12 to 30 carton atoms. Specific examples include mineral oil, petroleum jelly, squalene and isoparaffins.

Other embodiments of the compositions of the present invention comprise thickeners. A thickener will usually be present in amounts anywhere from 0.1 to 20% by weight, preferably from about 0.5% to 10% by weight of the composition. Exemplary thickeners are cross-linked polyacrylate materials available under the trademark CARBOPOL® from the B.F. Goodrich Co. Gums may be employed such as xanthan, carrageenan, gelatin, karaya, pectin and locust beans gum. Under certain circumstances the thickening function may be accomplished by a material also serving as a silicone or emollient. For instance; silicone gums in excess of 10 centistokes and esters such as glycerol stearate have dual functionality.

Powders may be incorporated into the cosmetic composition of the invention.

These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

Other adjunct minor components may also be incorporated into the cosmetic compositions. These ingredients may include coloring agents, opacifiers and perfumes. Amounts of these other adjunct minor components may range anywhere from 0.001% up to 20% by weight of the composition.

The composition of the invention may be used for topical application to human skin, as an agent for conditioning, moisturizing and smoothing the skin, increasing the flexibility and elasticity and preventing or reducing the appearance of wrinkled, lined or aged skin. Formulations of the present invention offer a response to the loss of skin tone and promotes benefits to effectively boost hydration and firmness of the surface layer of the skin, all while working to repair the underlying layers of the skin with antioxidants and other beneficial ingredients to help diminish the appearance of fine lines and wrinkles and to restore visible tone and elasticity. In some exemplary embodiments such anti-oxidants are specifically directed to inhibit arNOX.

In one embodiment a small quantity of the composition comprised of from about 1 to 1000 ml of active agent, is applied to the skin. In an exemplary embodiment, a quantity of composition comprising from about 1 to 100 ml of active agent is applied to the skin. This process may be repeated several times daily for any period of time. Preferably, the composition is applied to the skin once in the morning and once in the evening.

The topical skin care composition of the invention can be formulated as a lotion, a cream, a gel or the like. The composition can be packaged in a suitable container to suit its viscosity and intended use by the consumer. For example, a lotion or a cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The invention accordingly also provides a closed container containing a cosmetically acceptable composition as herein defined.

The following examples are offered by way of illustration and not by way of limitation.

### EXAMPLES

### Example 1

### Characterization of Narcissus tazetta Extracts

To test the hypothesis that N. tazetta may have arNOX inhibitory agents, bulbs of paper white (Narcissus tazetta) narcissus were obtained from a commercial provider (Brent and Becky's Bulbs, Gloucester, Va.). Water and ethanolic extracts were prepared which inhibited arNOX activity and augmented the inhibition when combined with Narcissus tazetta powder (Xian Aojing Science and Technology Developing CO., LTD, Xian, CN) of low activity. Augmentation of the powder derived from N. tazetta bulbs with SHAM (Sigma-Aldrich, St. Louis, Mo.) is shown in FIG. 1a. Confirmation of the ability of the naractins to augment arNOX inhibition was made by following the same protocol, but using salicin (Sigma-Aldrich) and N. tazetta powder derived from the flower (Xian Aojing Science and Technology Developing CO., LTD, Xian, CN) to augment the arNOX inhibition, FIG. 1b. Comparisons were with similar extracts of Narcissus pseudo-narcissus (daffodil) and Narcissus jonquilla (jonquil) extracts which lacked arNOX inhibitory activity. As illustrated in FIG. 2, activity of the N. tazetta extracts was correlated with a red coloration enhanced by the addition of ferric iron, a characteristic of naturally-occurring hydroxamates, as found in corn (maize) (FIG. 3) and oat (not shown) seedlings which also inhibit arNOX (FIG. 4). Further evidence for the potential ability of the active components of N. tazetta extracts to represent naturally-occurring hydroxamates was provided by thin layer chromatographic analyses (FIG. 5) and from spectral studies of their iron adducts and also from steeles of (the central vasculature of the maize root where the hydroxamates are concentrated and corresponding to the potentially hydroxamate-rich stem vasculature of the N. tazetta bulb (illustrated in FIG. 2) and of maize roots (FIG. 6) by comparison to a commercially available hydroxamate (salicyl hydroxamate (SHAM), Sigma-Aldrich)

SHAM, a known inhibitor of the alternative oxidase activity of plants, was tested for inhibition of arNOX activity and greater than 90% inhibition of arNOX activity of saliva from at 72 y male was obtained at a concentration of 1 mg/ml (FIG. 7). SHAM was also found to augment the inhibition of arNOX activity of partially active Narcissus tazetta powder (FIG. 1). The commercially available Narcissus tazetta powder (IBR-DORMIN®, Israel) does appear to contain low levels of a naturally-occurring hydroxamate possibly similar to SHAM but at levels much less than that found in extracts prepared from fresh Narcissus tazetta bulbs (FIGS. 5a and 5b).

### Example 2

### Identification of arNOX Inhibitors

To answer the question of whether or not the salicylic portion of the stabilized salicyl hydroxamate was important to inhibition of arNOX activity, salicylic acid (Naractin 2) was tested as an inhibitor of arNOX activity of saliva from a 72 y/o male and was found to inhibit arNOX activity (FIG. 8). Salicylic acid also gave a red color when reacted with ferric iron similar to that given by the hydroxamates. Esters of salicylic acid and/or aspirin did not inhibit arNOX activity. An aqueous extract of willow bark, a natural source of salicylates, was tested and found to inhibit arNOX as well. The glycoside salicin (Naractin 3), the major salicylate of willow bark, was subsequently tested and found to be active at a ten-fold lower concentration than either SHAM (Naractin 1) or salicylic acid (Naractin 2) (FIG. 9).

### Example 3

### Identification of Salicylate Producing Plants

A search was conducted to identify plants that were natural producers of salicylates. The search was conducted using the Natural Products database created at the University of Illinois in Chicago by Professor Norman Farnsworth (available at napralert.org). The search identified the following plants that produced salicylates in the various tissues identified: A. chinense (alangiaceae) dried leaf china, A. chinense (alangiaceae) dried leaf Japan (cult), A. platanifolium (alangiaceae) dried leaf France, A. platanifolium var. platanifolium (alangiaceae) dried leaf Japan, A. platanifolium var. trilobum (alangiaceae) dried leaf Japan, A. platanifolium var. trilobum (alangiaceae) dried leaf Japan, A. premnifolium (alangiaceae) leaf Japan, A. premnifolium (alangiaceae) dried stem Japan, Aspergillus niger (hyphomycetes) culture filtrate South Korea, betula alba (betulaceae) dried bark Germany, Bupleurum falcatum (apiaceae) suspension culture of seedling Japan, Catharanthus roseus (apocynaceae) suspension culture of leaf Japan, Chosenia bracteosa (salicaceae) dried bark Japan, Colchicum autumnale (liliaceae) fresh corm Japan, Crepis foetida (asteraceae) dried root Poland, Crepis rhoeadifolia (asteraceae) dried root Poland (cult), Datura inoxia (solanaceae) suspension culture of anthers Japan, D. inoxia (solanaceae) suspension culture of root Japan, Duboisia myoporoides (solanaceae) suspension culture of leaf Japan, Eleutherococcus setchuensis (araliaceae) dried stem china, Euphorbia salicifolia (euphorbiaceae) fresh entire plant Hungary, Filipendula ulmaria (rosaceae) dried aerial parts Europe, F. ulmaria (rosaceae) dried entire plant, F. ulmaria (rosaceae) dried entire plant Switzerland, F. ulmaria (rosaceae) dried flowers USSR, Foeniculum vulgare (apiaceae) commercial sample of fruit China, Gardenia jasminoides (rubiaceae) suspension culture of leaf Japan, Lithospermum erythrorhizon (boraginaceae) suspension culture of seedling Japan, Nicotiana tabacum (solanaceae) suspension culture of root Japan, P. alba (salicaceae) dried bark USA, P. balsamifera (salicaceae) dried bark USA-WI, P. balsamifera (salicaceae) fresh bark USA-WI, P. balsamifera (salicaceae) dried bark+twigs USA-WI, P. balsamifera (salicaceae) dried buds France, P. balsamifera (salicaceae) freeze-dried leaf USA-AK, P. balsamifera (salicaceae) fresh leaf USA-WI, P. balsamifera (salicaceae) oven dried leaf Finland (cult), P. balsamifera (salicaceae) dried trunk bark USA-WI, P. davidiana (salicaceae) dried bark china, P. davidiana (salicaceae) dried stem bark South Korea, P. deltoides (salicaceae) dried bark USA-WI, P. deltoides (salicaceae) fresh bark USA-WI, P. deltoides (salicaceae) fresh leaf USA-WI, P. deltoides var. deltoides (salicaceae) dried bark Canada (cult), P. deltoides var. occidentalis (salicaceae) dried bark Canada (cult), P. euphratica (salicaceae) dried bark china, P. euphratica (salicaceae) dried buds turkey, P. grandidentata (salicaceae) dried bark USA-WI, P. grandidentata (salicaceae) dried leaf USA-WI, P. heterophylla (salicaceae) dried bark USA-WI, P. lasiocarpa (salicaceae) dried buds England, P. maximowiczii (salicaceae) dried bark Japan, P. nigra (salicaceae) fresh bark Germany, P. nigra (salicaceae) dried leaf England (cult), P. nigra (salicaceae) dried leaf Germany, P. sieboldii (salicaceae) dried buds Japan, P. simonii (salicaceae) dried bark china, P. tacamahaca (salicaceae) dried bark USA-WI, P. tomentosa (salicaceae) dried bark china, P. tomentosa (salicaceae) entire plant china, P. tomentosa (salicaceae) dried leaf china, P. tremula (salicaceae) dried bark Germany, P. tremula (salicaceae) dried leaf Switzerland, P. tremula (salicaceae) oven dried leaf Finland, P. tremula (salicaceae) fungus infected stem bark France, P. tremula (salicaceae) dried twig Finland, P. tremuloides (salicaceae) dried bark USA, P. tremuloides (salicaceae) dried bark USA-WI, P. tremuloides (salicaceae) young entire plant, P. tremuloides (salicaceae) freeze-dried internodes USA-AK, P. tremuloides (salicaceae) dried leaf USA-WI, P. tremuloides triploid (salicaceae) fresh leaf USA-WI, P. tremuloides triploid type (salicaceae) dried bark USA-WI, P. trichocarpa (salicaceae) dried bark USA-WA, P. trichocarpa (salicaceae) dried bark USA-WI, P. trichocarpa (salicaceae) fresh leaf USA-WI, P. trichocarpa×P. deltoides (salicaceae) fresh leaf Belgium (cult), S. acutifolia (salicaceae) dried bark Russia, S. alba (salicaceae) dried bark, S. alba (salicaceae) dried bark France, S. alba (salicaceae) dried bark Germany, S. alba (salicaceae) dried bark USA, S. alba (salicaceae) dried bark USA-AR, S. alba (salicaceae) dried bark USA-UT, S. alba (salicaceae) oven dried leaf England, S. alba (salicaceae) oven dried leaf Finland (cult), S. alba cv. cardinalis (salicaceae) dried leaf England, S. alba sex female (salicaceae) dried bark Germany, S. alba sex male (salicaceae) dried bark Germany, S. alba×s. babylonica (s. sepula (salicaceae) oven dried leaf England, S. alba×s. fragilis (s. russellia (salicaceae) oven dried leaf England, S. alba×s. pentadra (s. ehrhardt (salicaceae) oven dried leaf England, S. alba×S. babylonica (salicaceae) entire plant USSR, S. americana (salicaceae) dried leaf England, S. arctica (salicaceae) dried leaf Iceland, S. aurita (salicaceae) dried bark Germany, S. aurita (salicaceae) oven dried leaf Finland, S. aurita sex female (salicaceae) dried bark Germany, S. babylonica (salicaceae) dried leaf India, S. babylonica (salicaceae) oven dried leaf England, S. babylonica cv. fardon weeping (salicaceae) dried leaf England, S. babylonica×s. fragilis (s. blanda) (salicaceae) oven dried leaf England, S. basfordiana (salicaceae) fresh leaf England, S. basfordiana (salicaceae) frozen leaf, S. caesia (salicaceae) fresh branches France, S. caesia (salicaceae) fresh leaf France, S. caesia (salicaceae) fresh stem France, S. calodendron (salicaceae) fresh leaf England, S. calodendron (salicaceae) frozen leaf, S. capitata (salicaceae) dried leaf china, S. caprea (salicaceae) dried bark Finland, S. caprea (salicaceae) dried bark Germany, S. caprea (salicaceae) dried bark Mexico, S. caprea (salicaceae) dried leaf Mexico, S. caprea (salicaceae) dried leaf USSR, S. caprea (salicaceae) oven dried leaf England, S. caprea (salicaceae) oven dried leaf Finland, S. caprea sex female (salicaceae) dried bark Germany, S. caprea sex male (salicaceae) dried bark Germany, S. caprea var. lanata (salicaceae) dried leaf England, S. caprea×s. lanata (s. balfourii) (salicaceae) oven dried leaf England, S. caprea×s. viminalis (s. serican (salicaceae) oven dried leaf England, S. chaenomeloides (salicaceae) dried leaf Japan, S. cinerea (salicaceae) dried bark Germany, S. cinerea (salicaceae) dried bark Switzerland, S. cinerea (salicaceae) oven dried flowers Finland, S. cinerea (salicaceae) dried leaf England, S. cinerea (salicaceae) oven dried leaf England, S. cinerea sex female (salicaceae) dried bark Germany, S. cinerea sex male (salicaceae) dried bark Germany, S. daphnoides (salicaceae) dried bark, S. daphnoides (salicaceae) dried bark Switzerland, S. daphnoides (salicaceae) fresh leaf England, S. daphnoides (salicaceae) frozen leaf, S. daphnoides clone 1 (salicaceae) dried twig Finland, S. daphnoides clone 2 (salicaceae) dried twig Finland, S. daphnoides ssp. cordaph (salicaceae) dried bark Madeira, S. daphnoides var. acutifolia (salicaceae) dried leaf England, S. fragilis (salicaceae) dried bark Germany, S. fragilis (salicaceae) dried leaf England, S. fragilis (salicaceae) dried leaf Germany, S. fragilis (salicaceae) fresh leaf England, S. fragilis (salicaceae) frozen leaf, S. fragilis (salicaceae) oven dried leaf England, S. fragilis (salicaceae) oven dried leaf Finland (cult), S. fragilis (salicaceae) dried twig Finland, S. fragilis sex male (salicaceae) dried bark Germany, S. fragilis sex male (salicaceae) dried leaf Germany, S. fragilis var. latifolia (salicaceae) fresh leaf England, S. fragilis var. latifolia (salicaceae) frozen leaf, S. fragilis×s. pentandra (s. meyeran (salicaceae) oven dried leaf England, S. fragilis×s. triandra (s. decipien (salicaceae) oven dried leaf England, S. geminata hybrid (salicaceae) dried leaf England, S. geminata hybrid (s. cinerea×s. vi (salicaceae) oven dried leaf England, S. gracilis var. textoris (salicaceae) oven dried bark Canada, S. gracilistyla (salicaceae) dried bark Japan, S. gracilistyla (salicaceae) dried leaf Japan, S. gracilistyloides (salicaceae) dried bark Japan, S. gymnolepis (salicaceae) dried bark Japan, S. hastata (salicaceae) dried bark Switzerland, S. herbacea×s. phylicifolla (s. moore (salicaceae) oven dried leaf England, S. incana (salicaceae) dried leaf England, S. koriyanagi (salicaceae) dried bark Japan, S. lapponum (salicaceae) oven dried leaf England, S. lapponum (salicaceae) oven dried leaf Finland, S. lapponum (salicaceae) dried twig Finland, S. lasiandra (salicaceae) freeze-dried leaf+stem USA-AK, S. lasiolepis (salicaceae) dried leaf USA-CA, S. matsudana (salicaceae) dried leaf china, S. myrsinifolia (salicaceae) dried bark Germany, S. myrsinifolia (salicaceae) dried leaf Finland (cult), S. myrsinifolia (salicaceae) dried leaf Germany, S. myrsinifolia (salicaceae) fresh leaf Finland, S. myrsinifolia (salicaceae) oven dried leaf Finland, S. myrsinifolia (salicaceae) dried stem Finland (cult), S. myrsinifolia (salicaceae) fresh stem Finland, S. myrsinifolia (salicaceae) dried twig Finland, S. myrsinifolia sex male (salicaceae) dried bark Germany, S. nigricans (salicaceae) dried leaf England, S. nigricans (salicaceae) dried leaf Switzerland, S. nigricans (salicaceae) fresh leaf England, S. nigricans (salicaceae) frozen leaf, S. orestera (salicaceae) dried leaf USA-CA, S. pentandra (salicaceae) dried bark Germany, S. pentandra (salicaceae) leaf USA, S. pentandra (salicaceae) dried leaf Germany, S. pentandra (salicaceae) fresh leaf England, S. pentandra (salicaceae) frozen leaf, S. pentandra (salicaceae) oven dried leaf England, S. pentandra (salicaceae) oven dried leaf Finland, S. pentandra (salicaceae) dried twig Finland, S. pentandra cv. lumley (salicaceae) dried leaf England, S. pentandra sex female (salicaceae) dried bark Germany, S. pentandra×s. fragilis (salicaceae) fresh leaf England, S. pentandra×s. fragilis (salicaceae) frozen leaf, S. pentandroides (salicaceae) dried leaf USSR, S. pentandroides (salicaceae) fresh root bark USSR, S. petiolaris (salicaceae) dried bark Canada, S. phylicifolia (salicaceae) dried leaf England, S. phylicifolia (salicaceae) oven dried leaf England, S. phylicifolia (salicaceae) oven dried leaf Finland, S. phylicifolia (salicaceae) dried twig Finland, S. phylicifolia×S. myrsinifolia (salicaceae) oven dried leaf Finland, S. purpurea (salicaceae) dried bark, S. purpurea (salicaceae) dried bark Germany, S. purpurea (salicaceae) dried bark Switzerland, S. purpurea (salicaceae) dried leaf Germany, S. purpurea (salicaceae) dried leaf Germany (cult), S. purpurea (salicaceae) dried leaf Switzerland, S. purpurea (salicaceae) fresh leaf, S. purpurea (salicaceae) fresh leaf England, S. purpurea (salicaceae) frozen leaf, S. purpurea (salicaceae) oven dried leaf England, S. purpurea sex female (salicaceae) dried bark Germany, S. purpurea sex female (salicaceae) dried leaf Germany, S. purpurea sex male (salicaceae) dried bark Germany, S. purpurea var. goldstones (salicaceae) dried leaf England, S. purpurea×s. triandra (s. leiophyl (salicaceae) oven dried leaf England, S. repens (salicaceae) dried bark France, S. repens (salicaceae) dried bark Germany, S. repens (salicaceae) dried leaf Germany, S. repens (salicaceae) fresh leaf England, S. repens (salicaceae) frozen leaf, S. repens sex female (salicaceae) dried bark Germany, S. repens sex male (salicaceae) dried bark Germany, S. rubra hybrid (salicaceae) dried leaf England, S. rubra hybrid (s. purpurea×vimi (salicaceae) oven dried leaf England, S. schwerinii (salicaceae) dried bark USSR, S. scouleriana (salicaceae) dried bark USA-UT, S. smithiana (salicaceae) dried leaf England, S. songarica (salicaceae) leaf USSR, S. songarica (salicaceae) dried leaf USSR, S. species (salicaceae) dried entire plant Switzerland, S. species (salicaceae) dried stem bark France, S. stipularis hybrid (s. viminalis×un (salicaceae) oven dried leaf England, S. tetrasperma (salicaceae) dried root Thailand, S. tetrasperma (salicaceae) dried stem bark Thailand, S. tremuloides (salicaceae) dried bark USA, S. triandra (salicaceae) dried bark Germany, S. triandra (salicaceae) oven dried leaf England, S. triandra cv. black maul (salicaceae) dried leaf England, S. triandra sex female (salicaceae) dried bark Germany, S. triandra sex male (salicaceae) dried bark Germany, S. triandra×s. viminalis (s. hippopha (salicaceae) oven dried leaf England, S. viminalis (salicaceae) dried bark Germany, S. viminalis (salicaceae) dried leaf England, S. viminalis (salicaceae) oven dried leaf England, S. viminalis (salicaceae) oven dried leaf Finland (cult), S. viminalis (salicaceae) dried twig Finland (cult), S. viminalis cv. aquatica (salicaceae) oven dried twig Finland (cult), S. viminalis sex female (salicaceae) dried bark Germany, Toisusu urbaniana (salicaceae) dried bark Japan, Viburnum henryi (caprifoliaceae) leaf, Viburnum prunifolium (caprifoliaceae) rootbark USA, Viburnum rhytidophyllum (caprifoliaceae) dried flowers Egypt, Viscum album e/S. alba (loranthaceae) leaf stem, France.

### Example 4

### Augmentation of arNOX Inhibitors

Not only was salicin active as a single agent (FIG. 9), it was also active in augmenting the arNOX inhibition of mixtures of other arNOX inhibiting agents from natural sources such as from Schizandra chinensis powder and from N. tazetta powder (FIG. 10a and 10b). As shown in FIG. 10a shows arNOX inhibition of a mixture of 4 mg/ml Schizandra powder plus N. tazetta extract (20 μl) with an added 1 mg/ml salicin. FIG. 1b illustrates the arNOX inhibition by a mixture of 4 mg/ml Schizandra powder plus 1 mg/ml N. tazetta powder in the presence of 1 mg/ml salicin. Of the three chemically pure “Naractins” (a term used to denote any one of several naturally occurring arNOX inhibitors either present in N. tazetta powder or capable of augmenting Narcissus tazetta powder to an inhibitory level comparable to that of the most active fresh N. tazetta extracts) identified and tested, salicin appeared to be the most promising. Salicin is stable, water soluble, non-irritating, relatively inexpensive and available from many commercial suppliers such as, for example Sigma-Aldrich, St. Louis, Mo. Further, augmentation of low activity N. tazetta powder is clearly shown in FIGS. 1a and 1b where both SHAM and salicin notably decreased arNOX activity.

### Example 5

### Characterization of arNOX

Superoxide Production By Buffy Coats: Buffy coats, a mixture of lymphocytes and platelets. Such buffy coats are commercially available from, for example, Rockland ImmunoChemicals (Gilbertsville, Pa.). The blood samples were maintained at 4° C. prior to collection and assay. Ca. 10⁷ cells were added to each assay. Cell numbers were determined using a hemocytometer.

Reduction of ferric cytochrome c by superoxide was employed as a standard measure of superoxide formation (Mayo, L. A. and Cumutte, J. (1990) Meth. Enzyme. 186, 567-575. 7. Butler, J, Koppenol, W. H. and Margollash, E. (1982) J. Biol. Chem. 257, 10747). This is a widely accepted method when coupled to superoxide dismutase inhibition for the measurement of superoxide generation. The assay consists of 150 μl serum or 40 μl buffy coats in PBSG buffer (8.06 g NaCl, 0.2 g KCl, 0.18 g Na₂HPO₄, 0.26 g KH₂PO₄, 0.13 g CaCl₂, 0.1 g MgCl₂ 1.35 g glucose dissolved in 1000 ml deionized water, adjusted to pH 7.4, filtered and stored at 4° C.) Rates were determined using an SLM Aminco DW-2000 spectrophotometer (Milton Roy, Rochester, N.Y., USA) in the dual wave length mode of operation with continuous measurements over 1 min every 1.5 min. After 45 min, test compounds were added and the reaction was continued for 45 min. After 45 min. a millimolar extinction coefficient of 19.1 cm⁻¹ was used for reduced ferricytochrome c. The results of the test compounds are provided in Table 1. Extracts were made of the compounds in water unless otherwise indicated.

Table 1 provides the results of some arNOX inhibition experiments.

TABLE 1

| | | | | INHIBITION (−) |
|---|---|---|---|---|
| | | | ArNOX ACTIVITY | or |
| | | | % OF NO | STIMULATION |
| SAMPLE | SOLVENT | CONCENTRATION | ADDITION | (+) |
| Broccoli extract | Water | 25 μg/ml | 85 | −15 |
| (1.5%) | | | | |
| Shiitake (10%) | Water | 25 μg/ml | 82 | −18 |
| Coleus | Water | 25 μg/ml | 106 | +6 |
| Centella | Water | 25 μg/ml | +3 | +3 |
| asiatica | | | | |
| Lotus leaf | Water | 25 μg/ml | 98 | −2 |
| extract | | | | |
| Artichoke | Water | 25 μg/ml | 98 | −2 |
| (15%) | | | | |
| Sea rose | Water | 25 μg/ml | 96 | −4 |
| Tangerine | Water | 25 μg/ml | 94 | −6 |
| Oenothera | Water | 25 μg/ml | 94 | −6 |
| biennis seed | | | | |
| Natural | Ethanol | 25 μg/ml | 62 | −38 |
| astaxanthin | | | | |
| Red orange | Ethanol | 25 μg/ml | 98 | −2 |
| Schisandra | Water | 20/2 μg/ml | 0/84 | −100/16 |
| chinensis | 30% Ethanol | | 20/94 | 80/6 |
| | 70% Ethanol | | 77/97 | 23/3 |
| Lonicera | Water | 25 μg/ml | 20 | −81 |
| japonica | | | | |
| Rhizoma | Water | 25 μg/ml | 0 | −100 |
| Fagopyrum | 70% EtOH | | | |
| cymosum | | | | |
| Rhizoma | | 25 μg/ml | ~50% | ~−50% |
| Fagopyrum | | | | |
| dibotrys | | | | |
| β-Carotene | Water | 25 μg/ml | 28 | −72 |
| | Ethanol | 25 μg/ml | 68 | −32 |
| | Ethanol | 2.5 μg/ml | 50 | −50 |
| | Ethanol | 0.25 μg/ml | 73 | −42 |

### Example 6

### Topical Cosmetic Preparations

An eight-week, split-face, controlled clinical usage study was conducted to screen four (4) prototype anti-aging formulations containing plant extracts with arNOX-inhibiting properties for their efficacy and tolerability versus two (2) vehicle controls. Efficacy was evaluated by clinical grading, bio-instrumentation measurements (Chroma Meter, Corneometer, Cutometer), and Self-Assessment Questionnaires. Tolerability was evaluated by irritation grading and monitoring for adverse events.

A total of 23 subjects completed study participation. Subjects qualified for study participation by having mild to moderate fine lines and coarse wrinkles in the periocular areas and hyperpigmentation on the right and left sides of the face. Subjects were assigned to two of the following test materials (one control and one test material) according to a randomization design:

Controls
A. arNOX Control Gel A (no label) AB-87-04A Colorless, transparent gel (12 subjects)
B. arNOX Control Gel B (red label) JZ-91-40 Colorless, transparent gel (contains glycerin) (11 subjects)

Test Materials:
1. arNOX Control Gel A w/Schizandra (non-encapsulated), N. tazetta extract and Salicin (green label) JZ 91-39, peach, transparent gel (6 subjects)
2. arNOX Control Gel A w/Schizandra (blue label) TL-90-59, colorless, transparent gel (6 subjects)
3. arNOX Control Gel B w/Schizandra (non-encapsulated), N. tazetta extract and Salicin (yellow label) TL-90-58 (contains glycerin) Peach, transparent gel (6 subjects)
4. arNOX Control Gel B w/Salicin (half yellow, half black label) KK-89-49, colorless, transparent gel (contains glycerin) (5 subjects)

Subjects were instructed to apply the assigned test material to the right or left side of the face and to apply the assigned Control to the opposite side of the face twice daily (in the morning and evening) after cleansing their faces.

Clinic evaluations were conducted at Baseline (Visit 1), Week 4 (Visit 2), and Week 8 (Visit 3). Subjects participated in the following clinical grading and instrumental procedures at each visit (unless otherwise indicated).

Efficacy/Performance Parameters

Subjects were clinically graded on the right and left sides of the face for the following parameters: fine wrinkles (periocular), coarse wrinkles (periocular), skin texture (cheeks), overall discoloration, brightness (cheeks), clarity of skin, pore size (forehead and nose area), pore distribution/structure, and overall skin radiance.

Irritation/Safety Parameter Grading

Subjects were clinically graded on the right and left sides of the face for objective irritation parameters (erythema, edema, scaling) and subjective irritation parameters (burning, stinging, itching, tightness, tingling).

Skin Surface Hydration Measurements

Skin surface hydration measurements were taken using the Corneometer® CM 825 (Courage+Khazaka, Germany) hydration analyzer. Measurements were taken (in triplicate) on the lower center of the left and right cheeks in order to quantify the moisture content of the stratum corneum.

Skin Luminance Measurements

Skin luminance measurements were made in triplicate using a Chroma Meter CR400 (Konica-Minolta, Japan) skin luminance analyzer and were taken on pigmented lesions (selected by the investigator) on the right and left sides of the face to instrumentally assess changes in skin color/tone. An additional Skin luminance measurement was taken on a non-pigmented (normal) area on one side of the face.

Skin Visco-Elasticity Measurements

A single visco-elasticity measurement was taken using the Cutometer® SEM 575 (Courage+Khazaka, Germany) visco-elasticity meter. Measurements were taken on the center of each subject's right and left cheeks in order to assess the visco-elastic properties of the skin.

Questionnaires

Subjects completed the following questionnaires at Week 4 and Week 8.
Subject Skin Change Evaluation questionnaire regarding changes in skin condition parameters since the start of the study
Subject Evaluation questionnaire regarding the current condition of skin condition parameters and test material attributes and tolerance

Overall, results of this study show that all test materials and controls produced significant improvements in the appearance of fine lines, tactile roughness, skin tone, and overall discoloration/hyperpigmentation, when compared to Baseline scores; test material 1, arNOX Control Gel A w/Schizandra (non-encapsulated), N. tazetta extract and Salicin (green label) JZ-91-39 and test material 3. arNOX Control Gel B w/Schizandra (non-encapsulated), N. tazetta extract and Salicin (yellow label) TL-90-58 (contains glycerin) improved the appearance of coarse wrinkles. There were no significant increases in objective or subjective irritation with any of the test materials or controls.

Skin luminance measurements show that only test material 1, arNOX Control Gel A w/Schizandra (non-encapsulated), N. tazetta extract and Salicin (green) JZ-91-39 produced a significant reduction in b* values at the non-pigmented site at Week 8. Skin luminance b* values taken at the pigmented lesion sites show that test material 1. arNOX Control Gel A w/Schizandra (non-encapsulated), N. tazetta extract and Salicin (green) JZ-91-39 was superior to test material 2. arNOX Control Gel A w/Schizandra (blue) TL-90-59 and test material 3. arNOX Control Gel B w/Schizandra (non-encapsulated), N. tazetta extract and Salicin (yellow) TL90-58 (contains glycerin) at reducing lesion darkness. Skin surface hydration measurements show that all of the test materials and controls significantly improved skin hydration at Week 4. Visco-elasticity measurement did not show any meaningful differences between the test materials and controls.

Informed Consent

Written informed consent conforming to 21 CFR 50.25 (Code of Federal Regulations) was obtained from each subject prior to enrollment in the study. The original, signed Informed Consent Agreement for each subject participating in the study will be retained in the study file. Each subject received a signed copy of the agreement. (Please see Appendix IV for a sample form.)

Attrition

Twenty-three (23) subjects completed the study. Twenty-six (26) subjects enrolled to participate in the study, and three (3) subjects discontinued study participation due to the following reasons:
Voluntarily discontinued/adverse event: Subject 021
Failure to attend scheduled visit(s): Subjects 004 and 026
Voluntarily discontinued/scheduling conflict: 020, 022, 029
Failure to attend scheduled visit: 009, 034
Investigator discretion: 010

Subject Demographics

Twenty-three (23) female subjects completed the study. Table 2 provides a summary of the demographic information (age, ethnicity, and Fitzpatrick skin classification) for all subjects. For ethnicity and Fitzpatrick skin type, the number of subjects in each category is listed with the percentage of the subject population in parentheses. Ethnicity information was obtained from each subject's Eligibility and Health Questionnaire.

TABLE 2
Summary Of Demographic Information
Demographic Summary

| | | |
|---|---|---|
| Age | Mean Age ± Standard | 54.53 ± 5.23 |
| (Years) | Deviation | |
| | Minimum Age | 45.67 |
| | Maximum Age | 64.51 |
| Ethnicity | Asian | 1 (4.3%) |
| | Caucasian | 2 (8.7%) |
| | Hispanic | 20 (87.0%) |
| Fitzpatrick Skin | Type I | 5 (21.7%) |
| Classification | Type II | 12 (52.2%) |
| | Type III | 6 (26.1%) |

The Fitzpatrick Skin Classification is based on the skin's unprotected response to the first 30-45 minutes of sun exposure after a winter season without sun exposure. The categories of the skin types are as follows:
Type I. Always burns easily; never tans;
Type II. Always burns easily; tans minimally;
Type III. Burns moderately; tans gradually;
Type IV. Burns minimally; always tans well;
Type V. Rarely burns; tans profusely;
Type VI. Never burns; deeply pigmented;

Fitzpatrick reported an alternative classification system that is useful in assessing the degree of perioral and periorbital (periocular) wrinkles (rhytidosis):
Class I—Fine wrinkles;
Class II—Fine-to-moderately deep wrinkles and moderate number of wrinkle lines;
Class III—Fine-to-deep (coarse) wrinkles, numerous wrinkle lines, and redundant folds possibly present

### Example 7

### Procedures and Methods

Prior to the start of the study, prospective subjects participated in a three-day washout period, during which facial moisturizers were not applied to the face.

At Baseline (Visit 1), prospective subjects washed their faces and removed all make-up at least 30 minutes prior to arriving at the clinic. Prospective subjects brought their regular skin care regimen products for eligibility consideration. Subjects completed an Eligibility and Health Questionnaire and signed an Informed Consent Agreement, a Confidentiality Agreement, and a Photography Release Form.

Subjects participated in the following clinical grading procedures:

Efficacy/Performance Parameters

Subjects were clinically graded on the right and left sides of the face for the following parameters:
Fine Wrinkles—periocular area
Coarse Wrinkles—periocular area
Skin Texture (Visual Appearance)—cheeks
Tactile Roughness—cheeks
Overall Discoloration
Overall Skin Radiance

Results of the efficacy/performance parameter grading were recorded using the following 1 to 10 point scale:
1=Positive (1 to 3=Good/Desirable)
10=Negative (8 to 10=Undesirable)
Half-point scores were used as needed

Subjects qualified for continued study participation by having a mild to moderate score of 3 to 7 for periocular fine lines; 2 to 5 for periocular coarse wrinkles; and 2 to 7 for hyperpigmentation on the right and left sides of the face.

Irritation/Safety Parameter Grading

Subjects were clinically graded on the right and left sides of the face for objective irritation parameters (erythema, edema, scaling) and subjective irritation parameters (burning, stinging, itching, tightness, tingling). Results of the irritation grading were recorded using the following scale:
0=None
1=Mild
2=Moderate
3=Severe
Half-points were used as necessary
   Qualified subjects participated in the following instrumentation measurements:

### Example 8

### Skin Surface Hydration Measurements

Skin surface hydration measurements were taken using the Comeometer® CM 825 (Courage+Khazaka, Germany) hydration analyzer. Measurements were made in triplicate and were taken on the lower center of the left and right cheeks in order to quantify the moisture content of the stratum corneum. The measuring principle of the Corneometer® is based on capacitance measurement of a dielectric medium. Any change in the dielectric constant due to skin surface hydration variation alters the capacitance of a precision measuring capacitor. These measurements can detect very slightest changes in the hydration level of the skin with very high reproducibility. Readings are directly proportional to the skin's electrical capacitance and measurements increase as the skin becomes more hydrated.

### Example 9

### Skin Luminance Measurements

Skin luminance measurements were made in triplicate using a Chroma Meter CR400 (Konica-Minolta, Japan) skin luminance analyzer and were taken on pigmented lesions (selected by the investigator) on the right and left sides of the face. The Chroma Meter instrumentally (and objectively) assesses changes in skin color/tone. An additional Chroma Meter measurement was taken on a non-pigmented (normal) area on one side of the face. The Chroma Meter is a sensitive colorimeter that allows the setting and calibration of color-difference target colors. The Chroma Meter has a detachable head for easy and independent analysis of selected areas. The following values were recorded:
L*: Describes the relative brightness on a gray scale from black to white; values increase as the skin becomes brighter and lighter
a*: Describes the color hue ranging from red to green; values increase with improvements in skin vascularization, increased blood flow, and improved skin tone
b*: Describes the color hue ranging from blue to yellow; values typically decrease with skin lightening
   An additional Skin luminance measurement was taken on a non-pigmented (normal) area on one side of the face for each subject.

### Example 10

### Skin Visco-Elasticity Measurements

A single visco-elasticity measurement was taken using the Cutometer® SEM 575 (Courage+Khazaka, Germany) visco-elasticity meter. Measurements were taken on the center of each subject's right and left cheeks in order to assess the visco-elastic properties of the skin. The measuring principle is based on suction. Negative pressure is created in the device and the skin is drawn into the aperture of the probe. Inside the probe, the penetration depth is determined by a non-contact optical measuring system. The light intensity varies due to the penetration depth of the skin. The resistance of the skin to be sucked up by the negative pressure (firmness and its ability to return into its original position (elasticity) are displayed on the instrument as curves at the end of each measurement. Three-hundred (300) mbar of negative pressure was applied and released through an 8-millimeter (mm) probe. The movement of the skin into and out of the probe was recorded during the application and release of suction, and resiliency and extensibility were calculated.

Subjects were assigned to one of the following test material groups according to a randomization design:

Controls
A. arNOX Control Gel A (no label) AB-87-04A
B. arNOX Control Gel B (red label) JZ-91-40 (contains glycerin)

Test Materials
1. arNOX Control Gel A w/Schizandra (non-encapsulated), N. tazetta extract and Salicin (green label) JZ91-39
2. arNOX Control Gel A w/Schizandra (blue label) TL-90-59
3. arNOX Control Gel B w/Schizandra (non-encapsulated), N. tazetta extract and Salicin (yellow label) TL-90-58 (contains glycerin)
4. arNOX Control Gel B w/Salicin (half yellow, half black label) KK-89-49 (contains glycerin)

Subjects were instructed to apply the assigned test material to the right or left sides of the face and to apply the assigned control to the opposite side of the face according to the following usage instructions:
Apply a thin layer twice daily in the morning and evening after cleansing your face.
Moisturizers and make-up products may be applied after test material applications.

Subjects were provided with written usage instructions, a calendar of future visits, and a daily diary to record test material application times and comments.

Subjects returned to the clinic at Week 4 (Visit 2) and Week 8 (Visit 3), having washed their face and removed all make-up at least 30 minutes prior to each visit. The daily diaries and test materials were returned to the clinic and checked for usage compliance; new diaries (and test materials, if needed) were distributed at Week 4. Subjects received clinical grading and bio-instrumentation measurements (Chroma Meter, Comeometer and Cutometer) in accordance with the Baseline procedures. Each subject also completed a Subject Skin Change Evaluation Questionnaire and a Subject Evaluation Questionnaire regarding test material attributes, tolerance, and improvements in skin condition on the right and left sides of the face.

The formulations for each of the compositions are provided in Table 3, below.

TABLE 3

| arNOX - Control Gel A | | |
|---|---|---|
| Quantitative Product Formulation | | |
| Lab Formula Number: AB-87-04A | No label | |
| INCI | W/W % | Supplier |
| Water (Aqua) | 98.980000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Polysorbate 20 | 0.100000 | Symrise |
| Fragrance (Parfum) | 0.020000 | Ungerer |
| Total: | 100.000000 | |
| arNOX - Control Gel B | | |
| Quantitative Product Formulation | | |
| Lab Formula Number: JZ-91-40 | Red label | |
| INCI | W/W % | Supplier |
| Water (Aqua) | 85.050000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Polysorbate 20 | 0.100000 | Unigema |
| Fragrance (Parfum) | 0.020000 | Ungerer |
| Glycerin | 13.930000 | |
| Total: | 100.000000 | |
| arNOX - Control Gel A with Schizandra | | |
| (non-encapsulated), N. tazetta extract | | |
| and Salicin Quantitative Product | | |
| Formulation | | |
| Lab Formula Number: JZ-91-39 | Green label | |
| INCI | W/W % | Supplier |
| Water (Aqua) | 98.735000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Shizandra chinenis Fruit Extract | 0.040000 | Draco |
| Water (Aqua) | 0.140000 | House |
| Narcissus Tazetta Bulb Extract | 0.060000 | Symrise |
| Salicin | 0.005000 | Kaden Bio. |
| Polysorbate 20 | 0.100000 | Unigema |
| Fragrance (Parfum) | 0.020000 | Ungerer |
| Total: | 100.000000 | |
| arNOX - Control Gel A with Salicin | | |
| Quantitative Product Formulation | | |
| Lab Formula Number: TL-90-59 | Blue label | |
| INCI | W/W % | |
| Water (Aqua) | 98.980000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Salicin | 0.005000 | Kaden Bio |
| Polysorbate 20 | 0.100000 | Unigema |
| Fragrance (Parfum) | 0.020000 | Ungerer |
| Total: | 100.005000 | |
| arNOX - Control Gel B with Schizandra | | |
| (non- encapsulated), N. tazetta and | | |
| Salicin Quantitative Product Formulation | | |
| Lab Formula Number: TL-90-58 | Yellow label | |
| INCI | W/W % | |
| Water (Aqua) | 84.805000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Water (Aqua) | 0.140000 | House |
| Narcissus Tazetta Bulb Extract | 0.060000 | Symrise |
| Salicin | 0.005000 | Kaden Bio |
| Glycerin | 13.930000 | House |
| Schizandra chinensis Fruit Extract | 0.040000 | Draco |
| Polysorbate 20 | 0.100000 | Unigema |
| Fragrance (Parfum) | 0.020000 | Ungerer |
| Total: | 100.000000 | |
| arNOX - Control Gel B with Salicin | | |
| Quantitative Product Formulation | | |
| Lab Formula Number: KK-89-49 | Yellow/Black label | |
| INCI | W/W % | |
| Water (Aqua) | 85.04500 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Salicin | 0.005000 | Kaden Bio |
| Glycerin | 13.930000 | House |
| Polysorbate 20 | 0.100000 | Unigema |
| Fragrance (Parfum) | 0.020000 | Ungerer |
| Total: | 100.000000 | |

**Noveon IP Holdings Corp. Cleveland, Ohio, U.S. Clariant, Corp. Charlotte, N.C., U.S. Angus Chemical Co., Buffalo Grove Il, U.S. Unigema, New Castle, DE, U.S. Symrise Inc., Teterboro, NJ Draco Natural Products, Inc., San Jose, CA, U.S.A. Xuancheng Baicao Plants Industry and Trade CO., LTD, Anhui, China

Subjects were provided with written usage instructions, a calendar of future visits, and a daily diary to record test material application times and comments.

Subjects returned to the clinic at Week 4 (Visit 2) and Week 8 (Visit 3). Subjects washed their faces and removed makeup at least 30 minutes prior to coming to the test facility for each visit. Subjects also brought their test materials to each visit for usage compliance checks. Subjects participated in the following procedures at each visit:
Efficacy/performance parameter grading
Irritation/safety parameter grading
Skin Surface Hydration (Corneometer®) measurements
Skin Luminence (Chroma Meter) measurements
Skin Visco-elasticity (Cutometer®) measurements
   Subjects also completed a Subject Skin Change Evaluation Questionnaire and a Subject Evaluation Questionnaire regarding test material attributes, tolerance, and improvements in skin condition parameters on the right and left sides of the face.

Daily diaries were returned to the clinic at each visit, and new diaries were distributed at Visits 2. Subjects returned test material units to the clinic at the completion of the study. Daily diaries were reviewed by clinic personnel and test material units were weighed at each visit to ensure compliance.

### Example 11

### Biostatistics and Data Management

Mean values for clinical grading and instrumentation measurements at Week 4 (Visit 2) and Week 8 (Visit 3) were statistically compared to mean Baseline (Visit 1) values using a paired t-test at the p≦0.05 significance level. Mean percent change from Baseline and incidence of improvement were calculated for all attributes. Comparisons were made among the test materials and controls using analysis of variance (ANOVA) with paired comparisons (Fisher's LSD).

Self-Assessment Questionnaires completed by subjects at Week 4 and Week 8 were tabulated and a top box analysis was performed.

### Example 12

### Topical Application Data

At Baseline (Visit 1), Week 4 (Visit 2), and Week 8 (Visit 3), subjects had clinical grading and bio-instrumentation measurements (Chroma Meter, Corneometer and Cutometer) performed on the face. Table 4 presents the results for each test material and control. Mean values at Week 4 and Week 8 are statistically compared to mean Baseline values for significant differences. The average percent change from Baseline is listed in parentheses.

TABLE 4

| | | | Baseline | Week 4 | | | Week 8 | | |
|---|---|---|---|---|---|---|---|---|---|
| | | | A. arNOX Control Gel A (no label) AB-87-04A | | | | | | |
| | | | (n = 12) No label | | | | | | |
| Efficacy/Performance | | Fine lines | 4.33 | 3.58 | [symbol] | (−17.3%) | 3.50 | [symbol] | (−19.2%) |
| Grading | | (periocular) | | | | | | | |
| | | Coarse wrinkles/skin | 3.63 | 3.25 | | (−10.3%) | 3.38 | | (−6.8%) |
| | | folds (periocular) | | | | | | | |
| | | Tactile roughness | 3.46 | 2.33 | [symbol] | (−32.5%) | 1.63 | [symbol] | (−53.0%) |
| | | (cheeks) | | | | | | | |
| | | Skin tone | 5.54 | 5.08 | [symbol] | (−8.2%) | 4.88 | [symbol] | (−12.0%) |
| | | Overall | 5.17 | 4.63 | [symbol] | (−10.4%) | 4.50 | [symbol] | (−12.9%) |
| | | discoloration/ | | | | | | | |
| | | hyperpigmentation | | | | | | | |
| | | Overall skin | 5.63 | 4.71 | [symbol] | (−16.2%) | 4.50 | [symbol] | (−20.0%) |
| | | radiance | | | | | | | |
| Irritation/Safety | | Erythema | 0.54 | 0.42 | | (−23.0%) | 0.25 | | (−53.8%) |
| Grading | | Edema | 0.00 | 0.00 | | | 0.00 | | |
| | | Scaling | 0.00 | 0.00 | | | 0.04 | | |
| | | Burning | 0.08 | 0.08 | | (0.0%) | 0.00 | | (−100.0%) |
| | | Stinging | 0.00 | 0.33 | | | 0.00 | | |
| | | Itching | 0.08 | 0.08 | | (0.0%) | 0.00 | | (−100.0%) |
| | | Tightness | 0.92 | 0.54 | [symbol] | (−40.9%) | 0.00 | [symbol] | (−100.0%) |
| | | Tingling | 0.00 | 0.00 | | | 0.00 | | |
| Chroma | Pigmented | L* | 63.28 | 62.60 | | (−1.0%) | 63.38 | | (0.1%) |
| Meter | Lesion | a* | 11.84 | 12.88 | | (8.7%) | 11.52 | | (−2.6%) |
| Measurements | | b* | 15.33 | 14.16 | [symbol] | (−7.6%) | 14.72 | | (−4.0%) |
| | Non-Pigmented | L* | 61.28 | 62.29 | | (1.6%) | 62.73 | [symbol] | (2.3%) |
| | Lesion | a* | 10.62 | 11.73 | [symbol] | (10.4%) | 11.89 | [symbol] | (11.9%) |
| | | b* | 14.92 | 14.28 | | (−4.3%) | 14.16 | | (−5.1%) |
| Corneometer Measurements | | | 39.31 | 62.69 | [symbol] | (59.5%) | 45.72 | | (16.3%) |
| Cutometer Measurements | | Biological Elasticity | 0.29 | 0.29 | | (1.2%) | 0.32 | | (8.3%) |
| | | Extensibility | 1.52 | 1.39 | | (−8.1%) | 1.01 | [symbol] | (−32.3%) |
| | | Pure Elasticity | 0.40 | 0.43 | | (5.2%) | 0.50 | [symbol] | (21.3%) |
| | | Resiliency | 0.68 | 0.66 | | (−3.3%) | 0.64 | | (−6.2%) |
| | | | B. arNOX Control Gel B (red) JZ-91-40 | | | | | | |
| | | | (contains glycerin) (n = 11) Red label | | | | | | |
| Efficacy/Performance | | Fine lines | 4.91 | 4.14 | [symbol] | (−15.7%) | 4.00 | [symbol] | (−18.5%) |
| Grading | | (periocular) | | | | | | | |
| | | Coarse wrinkles/skin | 4.05 | 3.86 | | (−4.4%) | 3.38 | | (−4.4%) |
| | | folds (periocular) | | | | | | | |
| | | Tactile roughness | 3.59 | 2.32 | [symbol] | (−35.4%) | 2.00 | [symbol] | (−44.3%) |
| | | (cheeks) | | | | | | | |
| | | Skin tone | 6.59 | 6.14 | [symbol] | (−6.8%) | 5.91 | [symbol] | (−10.3%) |
| | | Overall | 5.64 | 5.32 | | (−5.6%) | 5.45 | | (−3.2%) |
| | | discoloration/ | | | | | | | |
| | | hyperpigmentation | | | | | | | |
| | | Overall skin | 6.59 | 5.32 | | (−5.6%) | 5.45 | | (−3.2%) |
| | | radiance | | | | | | | |
| Irritation/Safety | | Erythema | 0.82 | 0.27 | [symbol] | (−66.6%) | 0.32 | [symbol] | (−61.1%) |
| Grading | | Edema | 0.00 | 0.00 | | | 0.00 | | |
| | | Scaling | 0.00 | 0.00 | | | 0.00 | | |
| | | Burning | 0.00 | 0.00 | | | 0.00 | | |
| | | Stinging | 0.00 | 0.00 | | | 0.00 | | |
| | | Itching | 0.27 | 0.09 | | (−66.6%) | 0.00 | | (−100.0%) |
| | | Tightness | 1.36 | 0.32 | [symbol] | (−76.6%) | 0.36 | [symbol] | (−73.3%) |
| | | Tingling | 0.00 | 0.00 | | | 0.00 | | |
| Chroma | Pigmented | L* | 61.76 | 61.67 | [symbol] | (−0.1%) | 61.70 | | (0.0%) |
| Meter | Lesion | a* | 11.38 | 12.16 | | (6.8%) | 12.48 | [symbol] | (−9.6%) |
| Measurements | | b* | 16.37 | 15.52 | [symbol] | (−5.2%) | 15.73 | | (−3.9%) |
| | Non- | L* | 61.25 | 61.91 | | (1.0%) | 62.47 | | (1.9%) |
| | Pigmented | a* | 10.68 | 11.07 | | (3.5%) | 11.64 | [symbol] | (8.9%) |
| | Lesion | b* | 15.49 | 14.72 | [symbol] | (−4.9%) | 14.46 | [symbol] | (−6.6%) |
| Corneometer Measurements | | | 42.91 | 75.27 | [symbol] | (75.4%) | 47.67 | | (11.0%) |
| Cutometer | | Biological Elasticity | 0.25 | 0.29 | [symbol] | (16.2%) | 0.31 | | (24.4%) |
| Measurements | | Extensibility | 1.63 | 1.43 | [symbol] | (−12.0%) | 1.14 | [symbol] | (−29.9%) |
| | | Pure Elasticity | 0.35 | 0.43 | [symbol] | (23.5%) | 0.48 | [symbol] | (37.0%) |
| | | Resiliency | 0.65 | 0.71 | | (9.9%) | 0.63 | | (−2.0%) |
| | | | 1. arNOX Control Gel A w/Schizandra (non-encapsulated), | | | | | | |
| | | | N. tazetta extract and Salicin (green) | | | | | | |
| | | | JZ-91-39 (n = 6) Green label | | | | | | |
| Efficacy/Performance | | Fine lines | 4.00 | 3.25 | [symbol] | (−18.7%) | 2.58 | [symbol] | (−35.4%) |
| Grading | | (periocular) | | | | | | | |
| | | Coarse wrinkles/skin | 3.50 | 3.50 | | (0.0%) | 3.08 | [symbol] | (−11.9%) |
| | | folds (periocular) | | | | | | | |
| | | Tactile roughness | 3.83 | 2.58 | [symbol] | (−32.6%) | 1.50 | [symbol] | (−60.8%) |
| | | (cheeks) | | | | | | | |
| | | Skin tone | 4.92 | 4.33 | [symbol] | (−11.8%) | 3.75 | [symbol] | (−23.7%) |
| | | Overall | 5.00 | 4.25 | [symbol] | (−15.0%) | 3.67 | [symbol] | (−26.6%) |
| | | discoloration/ | | | | | | | |
| | | hyperpigmentation | | | | | | | |
| | | Overall skin | 5.58 | 4.50 | [symbol] | (−19.4%) | 3.67 | [symbol] | (−34.3%) |
| | | radiance | | | | | | | |
| Irritation/Safety | | Erythema | 0.50 | 0.08 | | (−83.3%) | 0.08 | ** | (−83.3%) |
| Grading | | Edema | 0.00 | 0.00 | | | 0.00 | | |
| | | Scaling | 0.00 | 0.00 | | | 0.00 | | |
| | | Burning | 0.00 | 0.00 | | | 0.00 | | |
| | | Stinging | 0.00 | 0.00 | | | 0.00 | | |
| | | Itching | 0.00 | 0.00 | | | 0.00 | | |
| | | Tightness | 0.83 | 0.25 | [symbol] | (−70.0%) | 0.00 | [symbol] | (−100.0%) |
| | | Tingling | 0.00 | 0.00 | | | 0.00 | | |
| Chroma | Pigmented | L* | 61.18 | 60.37 | | (−1.3%) | 62.42 | | (2.0%) |
| Meter | Lesion | a* | 11.98 | 13.15 | | (9.7%) | 12.45 | | (3.8%) |
| Measurements | | b* | 16.00 | 14.89 | [symbol] | (−6.9%) | 15.07 | | (−5.8%) |
| | Non- | L* | 60.14 | 61.45 | | (2.1%) | 61.92 | | (2.9%) |
| | Pigmented | a* | 12.32 | 13.00 | | (5.5%) | 13.37 | | (8.4%) |
| | Lesion | b* | 14.49 | 13.88 | | (−4.2%) | 13.96 | [symbol] | (−3.6%) |
| Corneometer Measurements | | | 40.28 | 64.44 | [symbol] | (60.0%) | 42.11 | | (4.5%) |
| Cutometer | | Biological Elasticity | 0.28 | 0.31 | | (9.8%) | 0.30 | | (8.8%) |
| Measurements | | Extensibility | 1.64 | 1.36 | [symbol] | (−17.4%) | 1.04 | [symbol] | (−37.0%) |
| | | Pure Elasticity | 0.38 | 0.43 | | (13.7%) | 0.47 | | (25.7%) |
| | | Resiliency | 0.71 | 0.75 | | (5.2%) | 0.58 | | (−17.6%) |
| | | | 2. arNOX Control Gel A w/Schizandra (blue) | | | | | | |
| | | | TL-90-59 (n = 6) Blue label | | | | | | |
| Efficacy/Performance | | Fine lines | 4.58 | 3.67 | [symbol] | (−20.0%) | 3.17 | [symbol] | (−30.9%) |
| Grading | | (periocular) | | | | | | | |
| | | Coarse wrinkles/skin | 3.25 | 2.83 | | (−12.8%) | 2.67 | | (−17.9%) |
| | | folds (periocular) | | | | | | | |
| | | Tactile roughness | 3.33 | 2.00 | [symbol] | (−41.0%) | 1.17 | [symbol] | (−65.0%) |
| | | (cheeks) | | | | | | | |
| | | Skin tone | 6.17 | 5.17 | [symbol] | (−16.2%) | 5.58 | [symbol] | (−25.6%) |
| | | Overall | 5.83 | 4.67 | [symbol] | (−20.0%) | 4.17 | [symbol] | (−28.5%) |
| | | discoloration/ | | | | | | | |
| | | hyperpigmentation | | | | | | | |
| | | Overall skin | 5.75 | 4.75 | [symbol] | (−17.3%) | 4.25 | [symbol] | (−26.0%) |
| | | radiance | | | | | | | |
| Irritation/Safety | | Erythema | 0.67 | 0.50 | | (−25.0%) | 0.33 | | (−50.0%) |
| Grading | | Edema | 0.00 | 0.00 | | | 0.00 | | |
| | | Scaling | 0.00 | 0.00 | | | 0.00 | | |
| | | Burning | 0.17 | 0.17 | | (0.0%) | 0.17 | | (0.0%) |
| | | Stinging | 0.00 | 0.00 | | | 0.00 | | |
| | | Itching | 0.17 | 0.00 | | (−100.0%) | 0.00 | | (0.0%) |
| | | Tightness | 1.00 | 0.67 | | (−33.3%) | 0.00 | | (−100.0%) |
| | | Tingling | 0.00 | 0.00 | | | 0.00 | | |
| Chroma | Pigmented | L* | 62.17 | 62.35 | | (−0.2%) | 63.12 | | (1.5%) |
| Meter | Lesion | a* | 11.34 | 11.66 | | (2.8%) | 10.83 | | (−4.4%) |
| Measurements | | b* | 15.46 | 15.65 | | (1.2%) | 16.37 | | (6.0%) |
| | Non- | L* | 62.30 | 63.96 | | (2.6%) | 64.34 | [symbol] | (3.2%) |
| | Pigmented | a* | 9.81 | 9.60 | | (−2.1%) | 9.61 | | (−1.9%) |
| | Lesion | b* | 14.89 | 14.09 | | (−5.4%) | 14.35 | | (−3.6%) |
| Corneometer Measurements | | | 45.83 | 64.22 | [symbol] | (40.1%) | 48.56 | | (5.9%) |
| Cutometer | | Biological Elasticity | 0.28 | 0.29 | | (5.3%) | 0.29 | | (5.2%) |
| Measurements | | Extensibility | 1.48 | 1.32 | | (−10.4%) | 1.33 | | (−9.8%) |
| | | Pure Elasticity | 0.38 | 0.44 | | (15.9%) | 0.49 | | (27.3%) |
| | | Resiliency | 0.64 | 0.64 | | (−0.5%) | 0.58 | | (−10.0%) |
| | | | 3. arNOX Control Gel B w/Schizandra (non-encapsulated), | | | | | | |
| | | | N. tazetta extract and Salicin (yellow) | | | | | | |
| | | | TL-90-58 (contains glycerin) (n = 6) Yellow label | | | | | | |
| Efficacy/Performance | | Fine lines | 4.42 | 3.25 | [symbol] | (−26.4%) | 3.08 | [symbol] | (−30.1%) |
| Grading | | (periocular) | | | | | | | |
| | | Coarse wrinkles/skin | 3.67 | 3.25 | [symbol] | (−11.3%) | 3.25 | [symbol] | (−11.3%) |
| | | folds (periocular) | | | | | | | |
| | | Tactile roughness | 3.75 | 2.42 | [symbol] | (−35.5%) | 2.00 | [symbol] | (−46.6%) |
| | | (cheeks) | | | | | | | |
| | | Skin tone | 6.42 | 5.67 | [symbol] | (−11.6%) | 5.17 | [symbol] | (−19.4%) |
| | | Overall | | | | | | | |
| | | discoloration/ | 5.42 | 4.75 | [symbol] | (−12.3%) | 4.33 | [symbol] | (−20.0%) |
| | | hyperpigmentation | | | | | | | |
| | | Overall skin | 6.50 | 5.58 | [symbol] | (−14.1%) | 4.67 | [symbol] | (−28.2%) |
| | | radiance | | | | | | | |
| Irritation/Safety | | Erythema | 0.67 | 0.25 | [symbol] | (−62.5%) | 0.17 | [symbol] | (−75.0%) |
| Grading | | Edema | 0.00 | 0.00 | | | 0.00 | | |
| | | Scaling | 0.00 | 0.00 | | | 0.00 | | |
| | | Burning | 0.00 | 0.00 | | | 0.00 | | |
| | | Stinging | 0.00 | 0.00 | | | 0.00 | | |
| | | Itching | 0.17 | 0.17 | | (0.0%) | 0.00 | | (−100.0%) |
| | | Tightness | 1.17 | 0.17 | [symbol] | (−85.7%) | 0.33 | [symbol] | (−71.4%) |
| | | Tingling | 0.00 | 0.00 | | | 0.00 | | |
| Chroma | Pigmented | L* | 61.81 | 61.54 | | (−0.4%) | 62.87 | | (1.7%) |
| Meter | Lesion | a* | 11.93 | 11.97 | | (0.3%) | 11.19 | | (−6.2%) |
| Measurements | | b* | 14.81 | 15.42 | | (4.1%) | 15.34 | | (3.5%) |
| | Non- | L* | 63.01 | 63.66 | | (1.0%) | 64.53 | | (2.4%) |
| | Pigmented | a* | 10.75 | 10.95 | | (1.8%) | 10.70 | | (−0.4%) |
| | Lesion | b* | 14.29 | 13.90 | | (−2.7%) | 13.62 | | (−4.6%) |
| Corneometer Measurements | | | 41.89 | 74.83 | [symbol] | (78.6%) | 46.56 | | (11.1%) |
| Cutometer | | Biological Elasticity | 0.30 | 0.31 | | (5.1%) | 0.27 | | (−9.0%) |
| Measurements | | Extensibility | 1.48 | 1.42 | | (−3.8%) | 1.31 | | (−11.1%) |
| | | Pure Elasticity | 0.41 | 0.45 | | (9.6%) | 0.44 | | (6.0%) |
| | | Resiliency | 0.70 | 0.65 | | (−7.5%) | 0.51 | | (−28.1%) |
| | | | 4. arNOX Control Gel B w/ Salicin | | | | | | |
| | | | (half-yellow, half-black) | | | | | | |
| | | | KK-89-49 (contains glycerin) (n = 5) Yellow/Black label | | | | | | |
| Efficacy/Performance | | Fine lines | 5.40 | 4.40 | [symbol] | (−18.5%) | 3.80 | [symbol] | (−29.6%) |
| Grading | | (periocular) | | | | | | | |
| | | Coarse wrinkles/skin | 3.90 | 3.90 | | (−0.0%) | 3.80 | | (−2.5%) |
| | | folds (periocular) | | | | | | | |
| | | Tactile roughness | 3.50 | 2.30 | [symbol] | (−34.2%) | 1.70 | [symbol] | (−51.4%) |
| | | (cheeks) | | | | | | | |
| | | Skin tone | 6.80 | 6.00 | [symbol] | (−11.7%) | 5.60 | [symbol] | (−17.6%) |
| | | Overall | 5.90 | 4.80 | [symbol] | (−18.6%) | 4.70 | [symbol] | (−20.3%) |
| | | discoloration/ | | | | | | | |
| | | hyperpigmentation | | | | | | | |
| | | Overall skin | 6.70 | 5.50 | [symbol] | (−17.9%) | 5.00 | [symbol] | (−25.3%) |
| | | radiance | | | | | | | |
| Irritation/Safety | | Erythema | 1.00 | 0.20 | [symbol] | (−80.0%) | 0.40 | | (−60.0%) |
| Grading | | Edema | 0.00 | 0.00 | | | 0.00 | | |
| | | Scaling | 0.00 | 0.00 | | | 0.00 | | |
| | | Burning | 0.00 | 0.00 | | | 0.00 | | |
| | | Stinging | 0.00 | 0.00 | | | 0.00 | | |
| | | Itching | 0.40 | 0.00 | | (−100.0%) | 0.00 | | (−100.0%) |
| | | Tightness | 1.60 | 0.50 | | (−68.7%) | 0.40 | [symbol] | (−75.0%) |
| | | Tingling | 0.00 | 0.00 | | | 0.00 | | |
| Chroma | Pigmented | L* | 61.06 | 62.83 | | (−2.8%) | 62.79 | | (2.8%) |
| Meter | Lesion | a* | 12.54 | 10.80 | | (−13.8%) | 12.49 | | (−0.3%) |
| Measurements | | b* | 16.75 | 16.81 | | (0.3%) | 16.52 | | (−1.3%) |
| | Non- | L* | 60.62 | 62.12 | | (2.4%) | 62.69 | | (3.4%) |
| | Pigmented | a* | 11.01 | 10.06 | | (−8.6%) | 11.18 | | (−1.5%) |
| | Lesion | b* | 17.11 | 16.09 | | (−6.0%) | 16.03 | | (−6.3%) |

| Corneometer Measurements | | 44.08 | 80.07 | [symbol] | (78.7%) | 62.20 | (38.8%) |
|---|---|---|---|---|---|---|---|
| Cutometer | Biological Elasticity | 0.30 | 0.36 | | (19.1%) | 0.31 | (3.0%) |
| Measurements | Extensibility | 1.61 | 1.23 | | (−23.3%) | 1.18 | (−26.4%) |
| | Pure Elasticity | 0.40 | 0.53 | [symbol] | (31.7%) | 0.47 | (15.2%) |
| | Resiliency | 0.70 | 0.73 | | (4.2%) | 0.61 | (−12.8%) |

[symbol]

Indicates a statistically significant (p ≦ 0.05) decrease compared to Baseline [symbol]
Indicates a statistically significant (p ≧ 0.05) increase compared to Baseline
*Subject 010 was removed from the Week 8 Cutometer analysis due to an error in instrument calibration at the rescheduled visit;
n-values equal 11 for Cutometer measurements at Week 8.

### Example 13

### Average Change from Baseline

Table 5 provides comparisons of the average change from the baseline for the clinical grading and instrumentation studies.

TABLE 5

| | | | | Green | Blue label | Yellow | Yellow/Black |
|---|---|---|---|---|---|---|---|
| | | | Red label | label Test | Test | label Test | Test |
| | | | Control B | Material 1 | Material 2 | Material 3 | Material 4 |
| | | | (n = 11) | (n = 6) | (n = 6) | (n = 6) | (n = 5) |
| | | Control A | | | | | |
| | | (n = 12) | | | | | |
| WEEK 4 | | | | | | | |
| Efficacy/Performance Grading | | | | | | | |
| Fine lines (periocular) | | (−17.3%) | (−15.7%) | (−18.7%) | (−20.0%) | (−26.4%) | (−18.5%) |
| Coarse wrinkles/skin folds | | (−10.3%) | (−4.4%) | (0.0%) | (−12.8%) | (−11.3%) | (0.0%) |
| (periocular) | | | | | | | |
| Tactile roughness (cheeks) | | (−32.5%) | (−35.4%) | (−32.6%) | (−40.0%) | (−35.5%) | (−34.2%) |
| Skin tone | | (−8.2%) | (−6.8%) | (−11.8%) | (−16.2%) | (−11.6%) | (−11.7%) |
| Overall discoloration/ | | (−10.4%) | (−5.6%) | (−15.0%) | (−20.0%) | (−12.3%) | (−18.6%) |
| hyperpigmentation | | | | | | | |
| Overall skin radiance | | (−16.2%) | (−13.7%) | (−19.4%) | (−17.3%) | (−14.1%) | (−17.9%) |
| Irritation/Safety Grading | | | | | | | |
| Erythema | | (−23.0%) | (−66.6%) | (−83.3%) | (−25.0%) | (−62.5%) | (−80.0%) |
| Edema | | — | — | — | — | — | — |
| Scaling | | — | — | — | — | — | — |
| Burning | | (0.0%) | — | — | (0.0%) | — | — |
| Stinging | | — | — | — | — | — | — |
| Itching | | (0.0%) | (−66.6%) | — | (−100.0%) | (0.0%) | (−100.0%) |
| Tightness | | (−40.9%) | (−76.6%) | (−70.0%) | (−33.3%) | (−85.7%) | (−68.7%) |
| Tingling | | — | — | — | — | — | — |
| Chroma Meter Measurements | | | | | | | |
| Pigmented Lesion | L* | (−1.0%) | (−0.1%) | (−1.3%) | (0.2%) | (−0.4%) | (2.8%) |
| | a* | (8.7%) | (6.8%) | (9.7%) | (2.8%) | (0.3%) | (−13.8%) |
| | b* | (−7.6%) | (−5.2%) | (−6.9%) | (1.2%) | (4.1%) | (0.3%) |
| Non-Pigmented Lesion | L* | (1.6%) | (1.0%) | (2.1%) | (2.6%) | (1.0%) | (2.4%) |
| | a* | (10.4%) | (3.5%) | (5.5%) | (−2.1%) | (1.8%) | (−8.6%) |
| | b* | (−4.3%) | (−4.9%) | (−4.2%) | (−5.4%) | (−2.7%) | (−6.0%) |
| Corneometer Measurements | | (59.5%) | (75.4%) | (60.0%) | (40.1%) | (78.6%) | (78.7%) |
| Cutometer Measurements | | | | | | | |
| Biological Elasticity | | (1.2%) | (16.2%) | (9.8%) | (5.3%) | (5.1%) | (19.1%) |
| Extensibility | | (−8.1%) | (−12.0%) | (−17.4%) | (−10.4%) | (−3.8%) | (−23.3%) |
| Pure Elasticity | | (5.2%) | (23.5%) | (13.7%) | (15.9%) | (9.6%) | (31.7%) |
| Resiliency | | (−3.3%) | (9.9%) | (5.2%) | (−0.5%) | (−7.5%) | (4.2%) |
| | | Control A | | | | | |
| | | (n = 12)* | | | | | |
| WEEK 8 | | | | | | | |
| Efficacy/Performance Grading | | | | | | | |
| Fine lines (periocular) | | (−19.2%) | (−18.5%) | (−35.4%) | (−30.9%) | (−30.1%) | (−29.6%) |
| Coarse wrinkles/skin folds | | (−6.8%) | (−4.4%) | (−11.9%) | (−17.9%) | (−11.3%) | (−2.5%) |
| (periocular) | | | | | | | |
| Tactile roughness (cheeks) | | (−53.0%) | (−44.3%) | (−60.8%) | (−65.0%) | (−46.6%) | (−51.4%) |
| Skin tone | | (−12.0%) | (−10.3%) | (−23.7%) | (−25.6%) | (−19.4%) | (−17.6%) |
| Overall discoloration/ | | (−12.9%) | (−3.2%) | (−26.6%) | (−28.5%) | (−20.0%) | (−20.3%) |
| hyperpigmentation | | | | | | | |
| Overall skin radiance | | (−20.0%) | (−21.3%) | (−34.3%) | (−26.0%) | (−28.2%) | (−25.3%) |
| Irritation/Safety Grading | | | | | | | |
| Erythema | | (−53.8%) | (−61.1%) | (−83.3%) | (−50.0%) | (−75.0%) | (−60.0%) |
| Edema | | — | — | — | — | — | — |
| Scaling | | — | — | — | — | — | — |
| Burning | | (−100.0%) | — | — | (0.0%) | — | — |
| Stinging | | — | — | — | — | — | — |
| Itching | | (−100.0%) | (−100.0%) | — | (0.0%) | (−100.0%) | (−100.0%) |
| Tightness | | (−100.0%) | (−73.3%) | (−100.0%) | (−100.0%) | (−71.4%) | (−75.0%) |
| Tingling | | — | — | — | — | — | — |
| Chroma Meter Measurements | | | | | | | |
| Pigmented Lesion | L* | (0.1%) | (0.0%) | (2.0%) | (1.5%) | (1.7%) | (2.8%) |
| | a* | (−2.6%) | (9.6%) | (3.8%) | (−4.4%) | (−6.2%) | (−0.3%) |
| | b* | (−4.0%) | (−3.9%) | (−5.8%) | (6.0%) | (3.5%) | (−1.3%) |
| Non-Pigmented Lesion | L* | (2.3%) | (1.9%) | (2.9%) | (3.2%) | (2.4%) | (3.4%) |
| | a* | (11.9%) | (8.9%) | (8.4%) | (−1.9%) | (−0.4%) | (1.5%) |
| | b* | (−5.1%) | (−6.6%) | (−3.6%) | (−3.6%) | (−4.6%) | (−6.3%) |

| | | | | | | |
|---|---|---|---|---|---|---|
| Corneometer Measurements | (16.3%) | (11.0%) | (4.5%) | (5.9%) | (11.1%) | (38.8%) |
| Cutometer Measurements | | | | | | |
| Biological Elasticity | (8.3%) | (24.4%) | (8.8%) | (5.2%) | (−9.0%) | (3.0%) |
| Extensibility | (−32.3%) | (−29.9%) | (−37.0%) | (−9.8%) | (−11.1%) | (−26.4%) |
| Pure Elasticity | (21.3%) | (37.0%) | (25.7%) | (27.3%) | (6.0%) | (15.2%) |
| Resiliency | (−6.2%) | (−2.0%) | (−17.6%) | (−10.0%) | (−28.1%) | (−12.8%) |

*Subject 010 was removed from the Week 8 Cutometer analysis due to an error in instrument calibration at the rescheduled visit;
n-values equal 11 for Cutometer measurements at Week 8.

### Example 14

### Comparison Between Groups

Comparisons, based on the average change from Baseline, were made among the treatments (test materials and controls) using analysis of variance (ANOVA) with paired comparisons (Fisher's LSD). The following rankings, provided in Table 6, illustrate the statistically significant (p≦0.05) differences among the experimental groups. Rankings are presented in order of the greatest to the least level of improvement, and parameters with no significant differences are not listed. The average change from Baseline is listed beneath each treatment.

TABLE 6

| Overall Discoloration - | Test | Test | Test | Test | | |
|---|---|---|---|---|---|---|
| Week 4 | Material 2 | Material 4 | Material 1 | Material 3 | Control A | Control B |
| (p = 0.0074) | (−1.17) | (−1.10) | (−0.75) | (−0.67) | (−0.54) | (−0.32) |
| Overall Discoloration - | Test | Test | Test | Test | | |
| Week 8 | Material 2 | Material 1 | Material 4 | Material 3 | Control A | Control B |
| (p = 0.0000) | (−1.67) | (−1.33) | (−1.20) | (−1.08) | (−0.67) | (−0.18) |
| Skin Tone - | Test | Test | Test | Test | | |
| Week 8 | Material 2 | Material 3 | Material 4 | Material 1 | Control B | Control A |
| (p = 0.0020) | (−1.58) | (−1.25) | (−1.20) | (−1.17) | (−0.68) | (−0.67) |
| Chroma Meter b* | | | | | | |
| Pigmented Lesion - | Test | | | Test | Test | Test |
| Week 8 | Material 1 | Control B | Control A | Material 4 | Material 3 | Material 2 |
| (p = 0.0388) | (−0.94) | (−0.65) | (−0.62) | (−0.23) | (0.53) | (0.94) |

### Example 15

### Results of Statistical Comparison for Instrumentation Measurements

Subjects were graded for fine lines (periocular), coarse wrinkles/skin folds (periocular), tactile roughness (cheeks), skin tone, overall discoloration/hyperpigmentation, and overall skin radiance on the right and left side of the face. Results of the clinical grading revealed the following significant improvements, when compared to Baseline shown in Table 7.

TABLE 7

| | | | | | Test | | Test | | Test | | Test | |
|---|---|---|---|---|---|---|---|---|---|---|---|---|
| | Control | | Control | | Material | | Material | | Material | | Material | |
| | A | | B | | 1 | | 2 | | 3 | | 4 | |
| | W4 | W8 | W4 | W8 | W4 | W8 | W4 | W8 | W4 | W8 | W4 | W8 |
| Fine Lines (periocular) | [symbol] | [symbol] | [symbol] | [symbol] | [symbol] | [symbol] | [symbol] | [symbol] | [symbol] | [symbol] | [symbol] | [symbol] |
| Coarse wrinkles/skin folds (periocular) | | | | | | [symbol] | | | [symbol] | [symbol] | | |
| Tactile roughness (cheeks) | [symbol] | [symbol] | [symbol] | [symbol] | [symbol] | [symbol] | [symbol] | [symbol] | [symbol] | [symbol] | [symbol] | [symbol] |
| Skin tone | [symbol] | [symbol] | [symbol] | [symbol] | [symbol] | [symbol] | [symbol] | [symbol] | [symbol] | [symbol] | [symbol] | [symbol] |
| Overall discoloration/hyperpigmentation | [symbol] | [symbol] | | | [symbol] | [symbol] | [symbol] | [symbol] | [symbol] | [symbol] | [symbol] | [symbol] |
| Overall skin radiance | [symbol] | [symbol] | [symbol] | [symbol] | [symbol] | [symbol] | [symbol] | [symbol] | [symbol] | [symbol] | [symbol] | [symbol] |

[symbol]

Indicates a statistically significant (p ≦ 0.05) decrease, improvement, compared to Baseline
W4 = Week 4
W8 = Week 8

### Example 16

### Irritation/Safety

Subjects were graded for erythema, edema, scaling, burning, stinging, itching, tightness, and tingling on the right and left side of the face.

Results of the clinical grading revealed significant improvements, when compared to Baseline as shown in Table 8 for example:

TABLE 8

| | | | | | Test | | Test | | Test | | Test | |
|---|---|---|---|---|---|---|---|---|---|---|---|---|
| | Control A | | Control B | | Material 1 | | Material 2 | | Material 3 | | Material 4 | |
| | W4 | W8 | W4 | W8 | W4 | W8 | W4 | W8 | W4 | W8 | W4 | W8 |
| Erythema | | | [symbol] | [symbol] | | | | | [symbol] | [symbol] | [symbol] | |
| Tightness | [symbol] | [symbol] | [symbol] | [symbol] | [symbol] | [symbol] | | [symbol] | [symbol] | [symbol] | | [symbol] |

[symbol]

Indicates a statistically significant (p ≦ 0.05) decrease, improvement, compared to Baseline
W4 = Week 4
W8 = Week 8

### Example 17

### Skin Luminence Measurements

Skin luminance was measured in triplicate using a skin luminance analyzer (Chroma Meter CR400, Konica-Minolta, Japan). Measurements were taken over pigmented lesions (selected by the Investigator) on the right and left sides of the face in order to instrumentally assess changes in skin color/tone. An additional skin luminance measurement was taken on a non-pigmented (normal) area on one side of the face.

Results of the skin luminance measurements are shown in Table 9 and revealed significant differences, when compared to baseline for example:

TABLE 9

| | | | | | | Test | | Test | | Test | | Test | |
|---|---|---|---|---|---|---|---|---|---|---|---|---|---|
| | | Control | | Control | | Material | | Material | | Material | | Material | |
| | | A | | B | | 1 | | 2 | | 3 | | 4 | |
| | | W4 | W8 | W4 | W8 | W4 | W8 | W4 | W8 | W4 | W8 | W4 | W8 |
| Pigmented | a* | | | | [symbol] | | | | | | | | |
| Lesion | b* | [symbol] | | [symbol] | | [symbol] | | | | | | | |
| Non- | L* | | [symbol] | | | | | | [symbol] | | | | |
| Pigmented | a* | [symbol] | [symbol] | | [symbol] | | | | | | | | |
| Lesion | b* | | | [symbol] | [symbol] | | [symbol] | | | | | | |

[symbol]

Indicates a statistically significant (p ≦ 0.05) decrease compared to Baseline [symbol]
Indicates a statistically significant (p ≦ 0.05) increase compared to Baseline
W4 = Week 4
W8 = Week 8

### Example 18

### Skin Surface Hydration Measurements

Skin surface hydration measurements were taken in triplicate (Comeometer CM 825, Courage+Khazaka, Germany). Measurements were taken on the lower center of the right and left cheeks in order to quantify the moisture content of the stratum corneum.

Results of the skin surface hydration measurements revealed significant increases (improvements) in moisturization for each treatment at Week 4, when compared to Baseline. No significant differences were found at Week 8.

### Example 19

### Skin Visco-Elasticity Measurements

A single visco-elasticity measurement was taken using a Cutometer MPA 580 (Courage+Khazaka, Germany). The measurement was taken on the center of each subject's right and left cheeks in order to assess the visco-elastic properties of the skin.

Results of the visco-elasticity measurement, shown in Table 10, revealed the following significant differences, when compared to Baseline, for example:

TABLE 10

| | | | | | Test | | Test | | Test | | Test | |
|---|---|---|---|---|---|---|---|---|---|---|---|---|
| | Control A | | Control B | | Material 1 | | Material 2 | | Material 3 | | Material 4 | |
| | W4 | W8 | W4 | W8 | W4 | W8 | W4 | W8 | W4 | W8 | W4 | W8 |
| Biological Elasticity | | | [symbol] | | | | | | | | | |
| Extensibility | | [symbol] | [symbol] | [symbol] | [symbol] | [symbol] | | | | | | |
| Pure Elasticity | | [symbol] | [symbol] | [symbol] | | | | | | | [symbol] | |

[symbol]

Indicates a statistically significant (p ≦ 0.05) decrease compared to Baseline [symbol]
Indicates a statistically significant (p ≦ 0.05) increase compared to Baseline
W4 = Week 4
W8 = Week 8

### Example 20

### Overall Conclusions

Results of this pilot study show that all test materials and controls produced significant improvements in the appearance of fine lines, tactile roughness, skin tone, and overall discoloration/hyperpigmentation, when compared to Baseline scores. Of special note, test material 1, arNOX Control Gel A w/Schizandra (non-encapsulated), N. tazetta extract and Salicin (green label) JZ-91-39 and test material 3, arNOX Control Gel B w/Schizandra (non-encapsulated), Narcissus tazetta extract and Salicin (yellow label) TL-90-58 (contains glycerin) even had a positive effect on the appearance of coarse wrinkles showing significant improvements at week 4 (test material 3, yellow label) and week 8 (test materials 1, green label, and 3, yellow label). There were no significant increases in objective or subjective irritation with any of the test materials or controls.

Skin luminance measurements show that only test material 1, arNOX Control Gel A w/Schizandra (non-encapsulated), N. tazetta extract and Salicin (green label) JZ-91-39 produced a significant reduction in b* values at the non-pigmented site at Week 8. Skin luminance b* values taken at the pigmented lesion sites show that test material 1. arNOX Control Gel A w/Schizandra (non-encapsulated), N. tazetta extract and Salicin (green label) JZ-91-39 was superior to test material 2. arNOX Control Gel A w/Schizandra (blue label) TL-90-59 and test material 3. arNOX Control Gel B w/Schizandra (non-encapsulated), N. tazetta extract and Salicin (yellow label) TL-90-58 (contains glycerin) at reducing lesion darkness.

Skin surface hydration measurements show that all of the test materials and controls significantly improved skin hydration at Week 4.

Skin visco-elasticity measurements did not show any meaningful differences between the test materials and controls.

ANOVA comparisons between the test materials and controls show that test material 1, (arNOX Control Gel A w/Schizandra (non-encapsulated), Narcissus tazetta extract and Salicin (green label) JZ-91-39) and test material 2, (arNOX Control Gel A w/Schizandra (blue label) TL-90-59) were superior to control A, (arNOX Control Gel A (no label) AB-87-04A) and control B, (arNOX Control Gel B (red label) JZ-91-40 (contains glycerin)) at improving overall discoloration at Week 4. Further, test material 1 and 2 were superior to control B, (arNOX Control Gel B (red label) JZ-91-40 (contains glycerin)) at Week 8. Test material 2, (arNOX Control Gel A w/Schizandra (blue label) TL-90-59), test material 3, (arNOX Control Gel B w/Schizandra (non-encapsulated), Narcissus tazetta extract and Salicin (yellow label) TL-90-58 (contains glycerin)), and test material 4, (arNOX Control Gel B w/Salicin (half yellow, half black label) KK-89-49 (contains glycerin)) were superior to control A, (arNOX Control Gel A (no label) AB-87-04A) and control B, (arNOX Control Gel B (red label) JZ-91-40 (contains glycerin)) at improving the appearance of at Week 8.

What is claimed is:

1. A topical composition useful for ameliorating the effects of aging comprising:
an effective amount of at least one arNOX inhibitory agent,
wherein the arNOX inhibitory agent is a naractin and wherein the naractin is effective in decreasing the effects of aging.

2. The composition of claim 1, wherein the naractin is derived from a plant extract.

3. The composition of claim 2, wherein the naractin is a salicylate or derivative thereof.

4. The composition of claim 2, wherein the naractin is purified from N. tazetta, willow, maize, crepis, poplar, viburnam, Aspergillus, alangium, birch, bupleurum, colchicum, spurge, filipendulum, gardenia, lithospermum, tobacco or mistletoe

5. The composition of claim 3, wherein the salicylate is salicin, salicylic acid, salicyl hydroxamate or combinations thereof.

6. The topical composition of claim 1, wherein the composition further includes a cosmetically or pharmaceutically acceptable carrier.

7. The topical composition of claim 1, wherein more than one arNOX inhibitory agent is present and the more than one arNOX inhibitory agent is in the form of a plant extract.

8. The topical composition of claim 7, wherein the plant is selected from broccoli, shitake, coleus rosemary, lotus, artichoke, sea rose tangerine, Oenothera biennis, astaxanthin, red orange, Schisandra chinensis, Lonicera, Fagopyrum, carrot, Narcissus tazetta, olive, willow, oat or maize.

9. The topical composition of claim 8, wherein the Lonicera is Lonicera japonica or Lonicera caprifolium.

10. The topical composition of claim 7, wherein the arNOX inhibitory agent is β-carotene or astaxanthin.

11. The topical composition of claim 7, wherein the naractin augments the effects of additional arNOX inhibitory agents.

12. The topical composition of claim 1, wherein the composition is administered as a cream, a milk, a lotion, a gel, a suspension of lipid or polymeric microspheres or nanospheres or vesicles, a soap, a shampoo or a sunscreen.

13. The topical composition of claim 1, wherein the effects of aging comprise: lines, wrinkles, hyperpigmentation, dehydration, loss of elasticity, angioma, dryness, itching, telangietasias, actinic purpura, seborrheic keratoses, lack of hydration, decrease in collagen or actinic keratoses.

14. The topical composition of claim 1, wherein the arNOX inhibitory agent is provided at a concentration of between about 5 μg/ml to about 500 μg/ml.

15. A method to inhibit the generation of reactive oxygen species by aging-related isoform of NADH oxidase, to ameliorate the effects of aging comprising:
administering a therapeutically effective amount of a composition comprising at least one of salicin, salicylic acid, salicyl hydroxamate to a patient in need thereof, such that generation of reactive oxygen species by aging-related isoform of NADH oxidase, is inhibited and wherein an effect of aging is ameliorated.

16. The method of claim 15, wherein the composition further comprises an extract from least one of broccoli, shitake, coleus rosemary, lotus, artichoke, sea rose tangerine, Oenothera biennis, astaxanthin, red orange, Schisandra chinensis, Lonicera, Fagopyrum, carrot, Narcissus tazetta, olive, willow, oat or maize.

17. The method of claim 15, wherein the composition is applied as a cream, a milk, a lotion, a gel, a suspension of lipid or polymeric microspheres or nanospheres or vesicles, a soap, a shampoo or a sunscreen.

18. The method of claim 15, wherein the effects of aging comprise: lines, wrinkles, hyperpigmentation, dehydration, loss of elasticity, angioma, dryness, itching, telangietasias, actinic purpura, seborrheic keratoses, lack of hydration, decrease in collagen or actinic keratoses.

19. A cosmetic method for ameliorating the effects of aging comprising applying to the skin a cosmetic composition comprising:
an effective amount of a naractin sufficient to inhibit arNOX,
wherein at least one arNOX mediated effect of aging is inhibited.

20. The method of claim 19, wherein the naractin is salicylate.

21. The method of claim 20, wherein the salicylate is salicin, salicyl hydroxamte, or salicylic acid.

22. The method of claim 19, wherein the cosmetic composition further comprises a plant extract comprising: carrot extract, olive extract, broccoli extract, shitake extract, coleus, extract rosemary extract, lotus extract, artichoke extract, sea rose extract tangerine extract, Oenothera biennis extract, red orange extract, Schisandra chinensis extract, Lonicera extract, Fagopyrum extract, willow extract, corn steele, oat steele or Narcissus tazetta extract.

23. The cosmetic method of claim 19, wherein the naractin is provided together with a cosmetically acceptable carrier.

24. The cosmetic method of claim 19, wherein the effects of aging comprise: lines, wrinkles, hyperpigmentation, dehydration, loss of elasticity, angioma, dryness, itching, telangietasias, actinic purpura, seborrheic keratoses, lack of hydration, decrease in collagen or actinic keratoses.

25. The cosmetic method of claim 19, wherein the naractin is applied at least once a day.

26. The cosmetic method of claim 19, wherein the naractin is provided in a cosmetic preparation at a concentration of between about 5 μg/ml to about 500 μg/ml.

27. The cosmetic method of claim 19, wherein the composition is administered as a cream, a milk, a lotion, a gel, a suspension of lipid or polymeric microspheres or nanospheres or vesicles, a soap, a shampoo or a sunscreen.

28. A kit for applying a cosmetic useful in ameliorating the effects of aging comprising:
at least one naractin; and
instruction for use.

29. The kit of claim 28, further comprising a cosmetic preparation suitable as a carrier for the at least one arNOX inhibitory plant extract.
